(19) United States
(12) Patent Application Publication
(10) Pub. No.: US 2026/0106038 A1
(43) Pub. Date: Apr. 16, 2026

(54) INFORMATION PROCESSING APPARATUS, INFORMATION PROCESSING METHOD, AND PROGRAM

(71) Applicant: Shiseido Company, Ltd., Chuo-ku, Tokyo (JP)

(72) Inventors: Nishiki UCHIBE, Tokyo (JP); Chieko MIZUMOTO, Tokyo (JP); Ryo YOKOTA, Tokyo (JP)

(73) Assignee: Shiseido Company, Ltd., Chuo-ku, Tokyo (JP)

(21) Appl. No.: 19/115,960

(22) PCT Filed: Oct. 26, 2023
(86) PCT No.: PCT/JP2023/038790

§ 371 Date: Mar. 27, 2025

(30) Foreign Application Priority Data
Oct. 27, 2022 (JP) 2022-172015

(51) Int. Cl. G16H 50/30

(52) U.S. Cl. CPC G16H 50/30

(57) ABSTRACT

An information processing apparatus includes an acquiring module configured to acquire core temperature log information on a history of a core temperature of a user, an estimating module configured to estimate skin condition of the user based on the core temperature log information, and a presenting module configured to present an estimation result of the skin condition to the user.

### TECHNICAL FIELD

The present invention relates to an information processing apparatus, an information processing method, and a program.

### BACKGROUND ART

Generally, various skin care actions (for example, massaging the skin or using skin care products) are performed to maintain good skin condition.

It is important to know the condition of your skin to select the most appropriate care.

Techniques for estimating skin condition based on images are known (see, for example, Japanese Laid-Open Patent Publication No. 2017-012337).

According to the technology of Japanese Laid-Open Patent Publication No. 2017-012337, the amount of glossy parts of the skin in a captured image and the amount of wrinkled parts of the skin in a captured image are calculated as skin evaluation indices, and a firmness evaluation unit evaluates the firmness of the subject's facial skin based on the skin evaluation index calculated by the skin index calculation unit.

### SUMMARY OF INVENTION

### Technical Problem The technology of Japanese Laid-Open Patent Publication No. 2017-012337 merely estimates skin condition based on an image of the skin (that is, the surface of the skin).

However, skin condition do not always appear on the surface of the skin.

In addition, to obtain an image of the skin, it is necessary to capture an image of the skin with a camera, which limits the number of samples.

Therefore, estimating skin condition based on a skin image is a limit to the accuracy of the estimation.

A purpose of the present subject matter is to improve the accuracy of estimating skin condition.

### Solution to Problem

One aspect of the present invention is
an information processing apparatus comprising:
an acquiring module configured to acquire core temperature log information on a history of a core temperature of a user;
an estimating module configured to estimate skin condition of the user based on the core temperature log information; and
a presenting module configured to present an estimation result of the skin condition to the user.

### BRIEF DESCRIPTION OF DRAWINGS

FIG. 1 is a block diagram showing a configuration of an information processing system of the present embodiment.

FIG. 2 is a functional block diagram of the information processing system of FIG. 1.

FIG. 3 is an explanatory diagram of an overview of the present embodiment.

FIG. 4 is a diagram showing a data structure of a user database of the present embodiment.

FIG. 5 is a diagram showing the data structure of a core temperature log database of the present embodiment.

FIG. 6 is a diagram showing a data structure of a skin care log database of the present embodiment.

FIG. 7 is a diagram showing the data structure of a physical condition log database of the present embodiment.

FIG. 8 is a diagram showing the data structure of a mental condition log database of the present embodiment.

FIG. 9 is a diagram showing a data structure of an organism log database of the present embodiment.

FIG. 10 is a diagram showing a data structure of an action log database of the present embodiment.

FIG. 11 is a sequence diagram of information processing of the present embodiment.

FIG. 12 is a diagram showing an example of a screen displayed in the information processing of FIG. 11.

FIG. 13 is a diagram illustrating an overview of the first modification.

FIG. 14 is a diagram illustrating an overview of the second modification.

FIG. 15 is a diagram illustrating an overview of the third modification.

FIG. 16 is a diagram illustrating an overview of the fourth modification.

FIG. 17 is a diagram illustrating an overview of the fifth modification.

FIG. 18 is a diagram illustrating an overview of the sixth modification.

FIG. 19 is a diagram illustrating an overview of the seventh modification.

FIG. 20 is a sequence diagram of information processing of the seventh modification.

FIG. 21 is a diagram showing examples of screens displayed in the information processing of FIG. 20.

FIG. 22 is a diagram illustrating an overview of the eighth modification.

FIG. 23 is a sequence diagram of information processing of the eighth modification.

FIG. 24 is a diagram showing an example of a screen displayed in the information processing of FIG. 23.

FIG. 25 is an explanatory diagram of an overview of the ninth modification.

FIG. 26 is a sequence diagram of information processing of the ninth modification.

FIG. 27 is a diagram showing examples of screens displayed in the information processing of FIG. 26.

### DESCRIPTION OF EMBODIMENTS

Hereinafter, the present embodiment is described in detail based on the drawings.

Note that, in the drawings for describing the present embodiments, the same components are denoted by the same reference sign in principle, and the repetitive description thereof is omitted.

### (1) Configuration of Information Processing System

The Configuration of information processing system will be described.

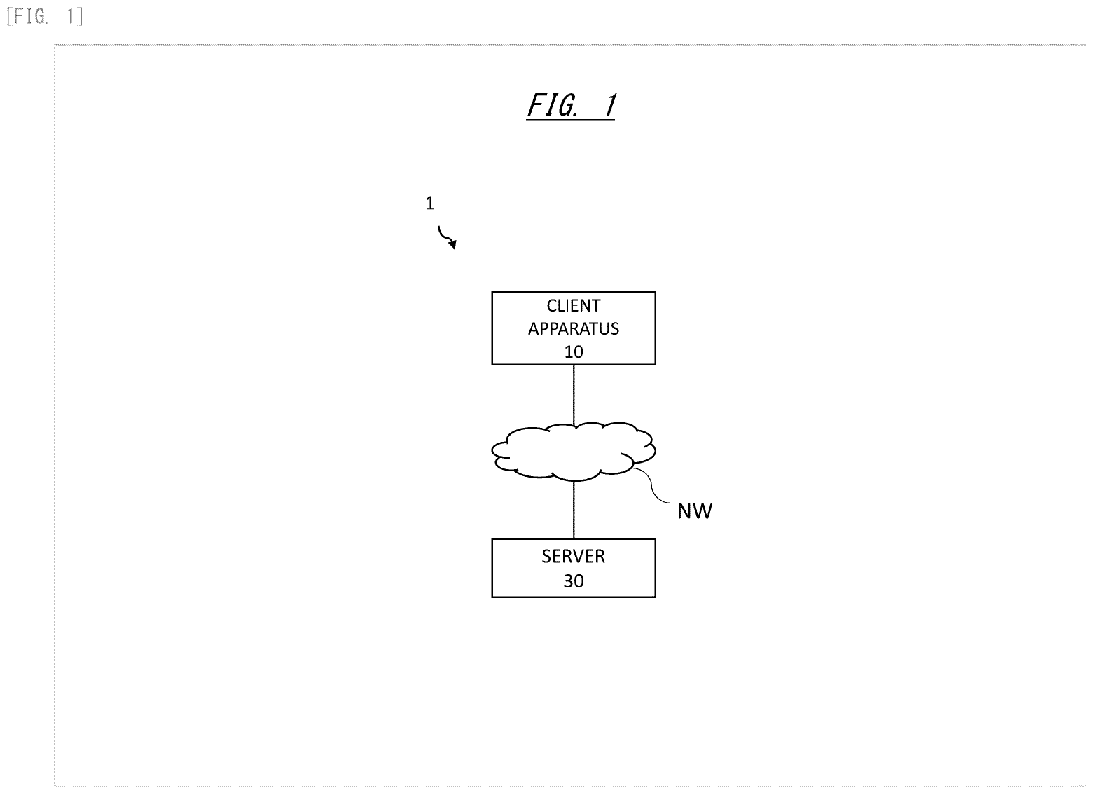

FIG. 1 is a block diagram showing the configuration of the information processing system of the present embodiment.

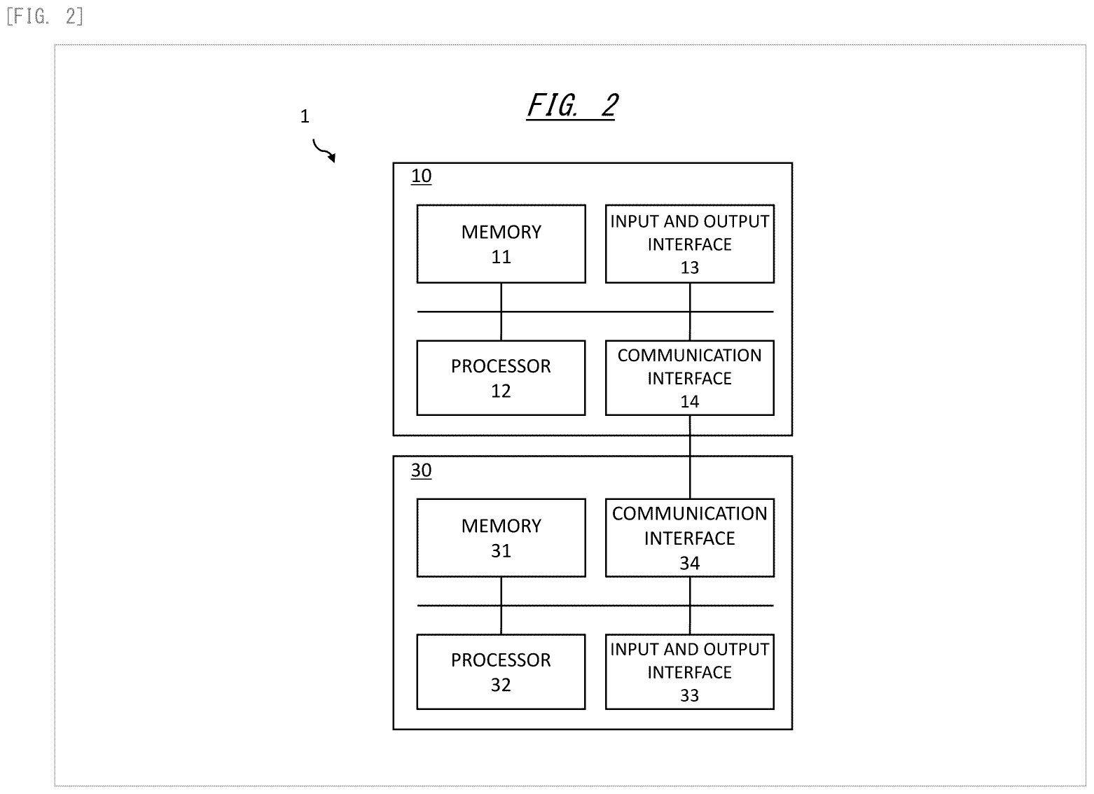

FIG. 2 is a functional block diagram of the information processing system of FIG. 1.

As shown in FIG. 1, the information processing system 1 includes a client apparatus 10 and a server 30.

The client apparatus 10 and the server 30 are connected via a network (for example, the Internet or an intranet) NW.

The client apparatus 10 is a computer (an example of an “information processing apparatus”) that transmits a request to the server 30.

The client apparatus 10 is, for example, a smart phone, a tablet terminal, or a personal computer.

The server 30 is a computer (an example of an “information processing apparatus”) that provides the client apparatus 10 with a response in response to a request sent from the client apparatus 10.

The server 30 is, for example, a web server.

### (1-1) Configuration of Client Apparatus

A configuration of the client apparatus 10 will be described.

As shown in FIG. 2, the client apparatus 10 includes a memory 11, a processor 12, an input and output interface 13, and a communication interface 14.

The memory 11 is configured to store programs and data.

The memory 11 is, for example, a combination of a ROM (read only memory), a RAM (random access memory), and a storage device(for example, a flash memory or a hard disk).

Programs include, for example, the following programs;
OS (Operating System) program; and
Programs of applications that execute information processing (for example, web browsers).

The data includes, for example, the following data:
Databases referenced in information processing; and
Data obtained by information processing.

The processor 12 is configured to implement the functions of the client apparatus 10 by activating programs stored in the memory 11.

The processor 12 is, for example, a CPU (Central Processing Unit), an ASIC (Application Specific Integrated Circuit), an FPGA (Field Programmable Gate Array), or a combination thereof.

The input and output interface 13 is configured to acquire a user's instruction from an input device connected to the client apparatus 10 and output information to an output device connected to the client apparatus 10.

The input device is, for example, a keyboard, a pointing device, a touch panel, or a combination thereof.

The output device is, for example, a display.

The communication interface 14 is configured to control communications between client apparatus 10 and server 30.

### (1-2) Configuration of Server

A configuration of the server 30 will be described.

As shown in FIG. 2, the server 30 includes a memory 31, a processor 32, an input and output interface 33, and a communication interface 34.

The memory 31 is configured to store a program and data.

The memory 31 is, for example, a combination of ROM, RAM, and storage device (for example, flash memory or hard disk).

Programs include, for example, the following programs:
OS program; and
Programs of applications that execute information processing.

The data includes, for example, the following data:
Databases referenced in the information processing; and
Data obtained by information processing.

The processor 32 is configured to implement the functions of the server 30 by activating programs stored in the memory 31.

Processor 32 is, for example, a CPU, ASIC, FPGA, or a combination thereof.

The input and output interface 33 is configured to acquire user's instruction from an input device connected to the server 30 and to output information to output devices connected to the server 30.

The input device is, for example, a keyboard, a pointing device, a touch panel, or a combination thereof.

The output device is, for example, a display.

Communication interface 34 is configured to control communications between server 30 and client apparatus 10.

### (2) Overview of Embodiment

An overview of the present embodiment will be described.

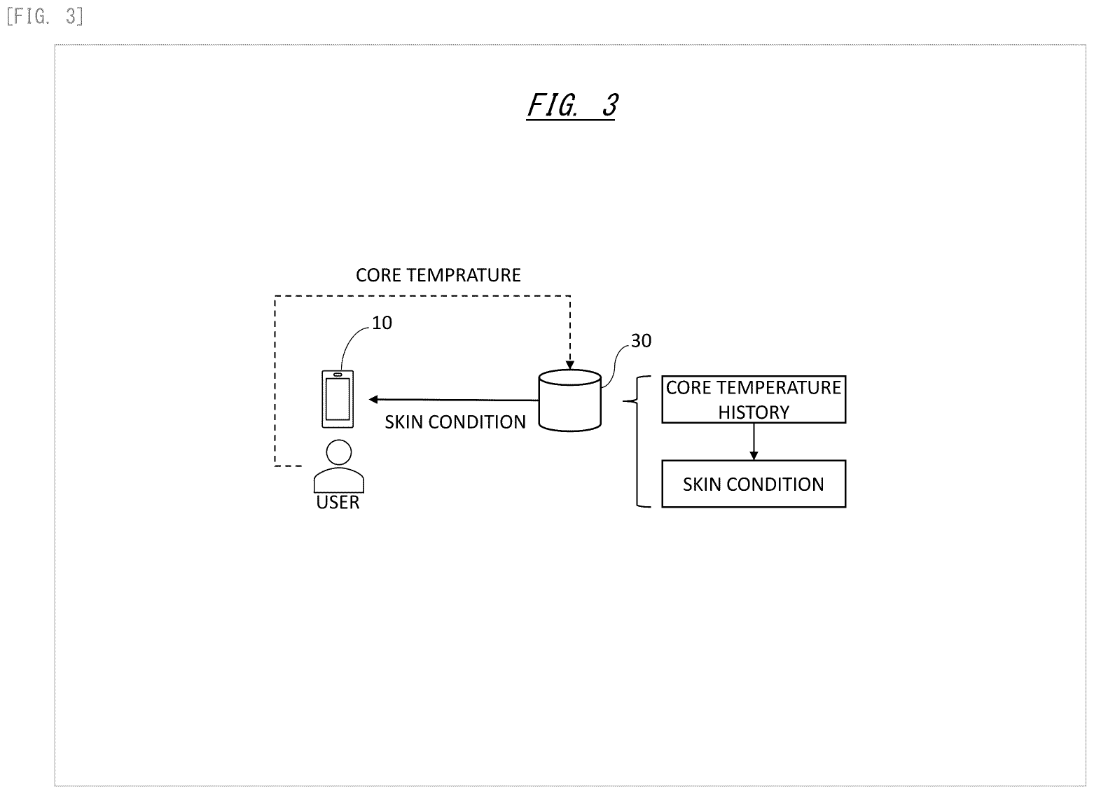

FIG. 3 is a diagram illustrating an overview of the present embodiment.

As shown in FIG. 3, the server 30 stores an user's core temperature history.

The server 30 estimates the user's skin condition based on the core temperature history.

The server 30 presents the estimation result (that is, the user's skin condition) to the user via the client apparatus 10.

### (3) Database

A database of the present embodiment will be described.

The following databases are stored in the memory 31.

### (3-1) User Database

The user database of the present embodiment will now be described.

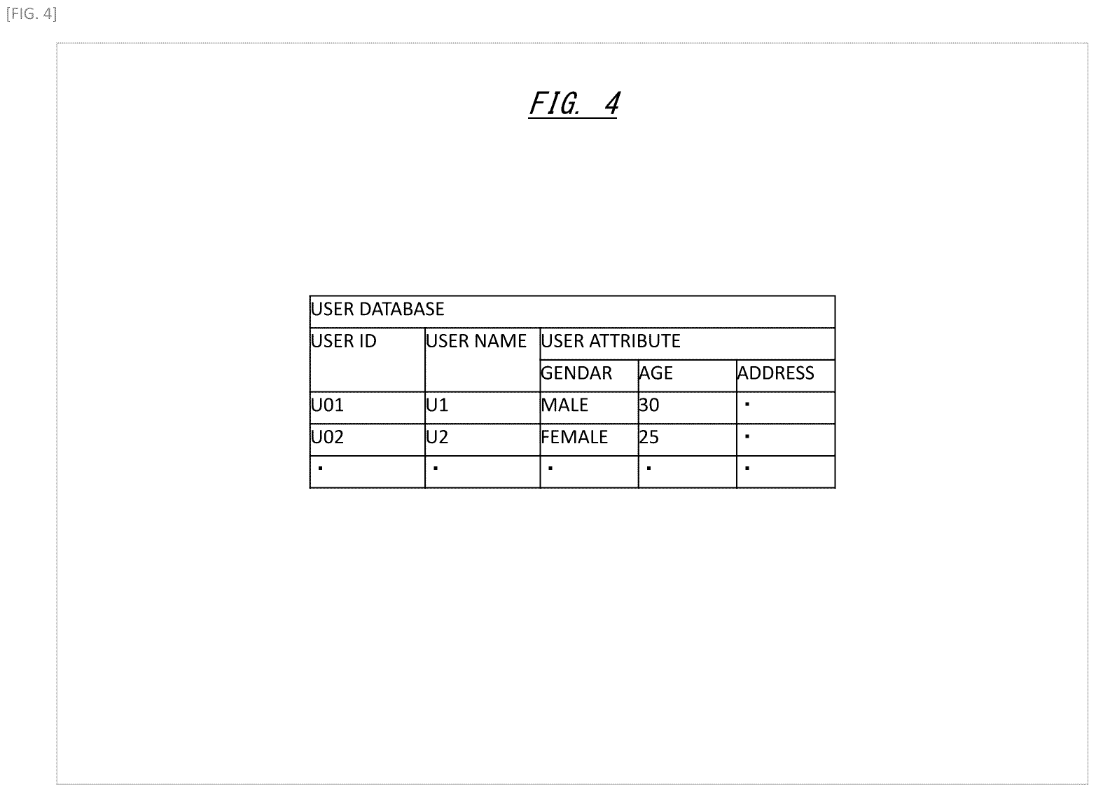

FIG. 4 is a diagram showing the data structure of the user database of the present embodiment.

The user database of FIG. 4 stores user information.

The user information is information on a user.

The user database includes a “user ID” field, a “user name” field, and a “user attribute”field.

Each field is associated with each other.

The “user ID” field stores user identification information.

The user identification information is information for identifying a user.

User name information is stored in the “user name” field.

The user name information is information on a user name (for example, a name, an account name, or a handle name).

The “user attribute” field stores user attribute information.

The user attribute information is information on the attributes of the user.

The “user attribute” field includes a “gender” field, and an “age” field, and an “address” field.

The “gender” field stores gender information.

The gender information is information on the gender of the user.

The “age” field stores age information.

The age information is information on the age of the user.

The “Address” field stores address information.

The address information is information on the address of the user's residence.

### (3-2) Core Temperature Log Database

The core temperature log database of the present embodiment will be described.

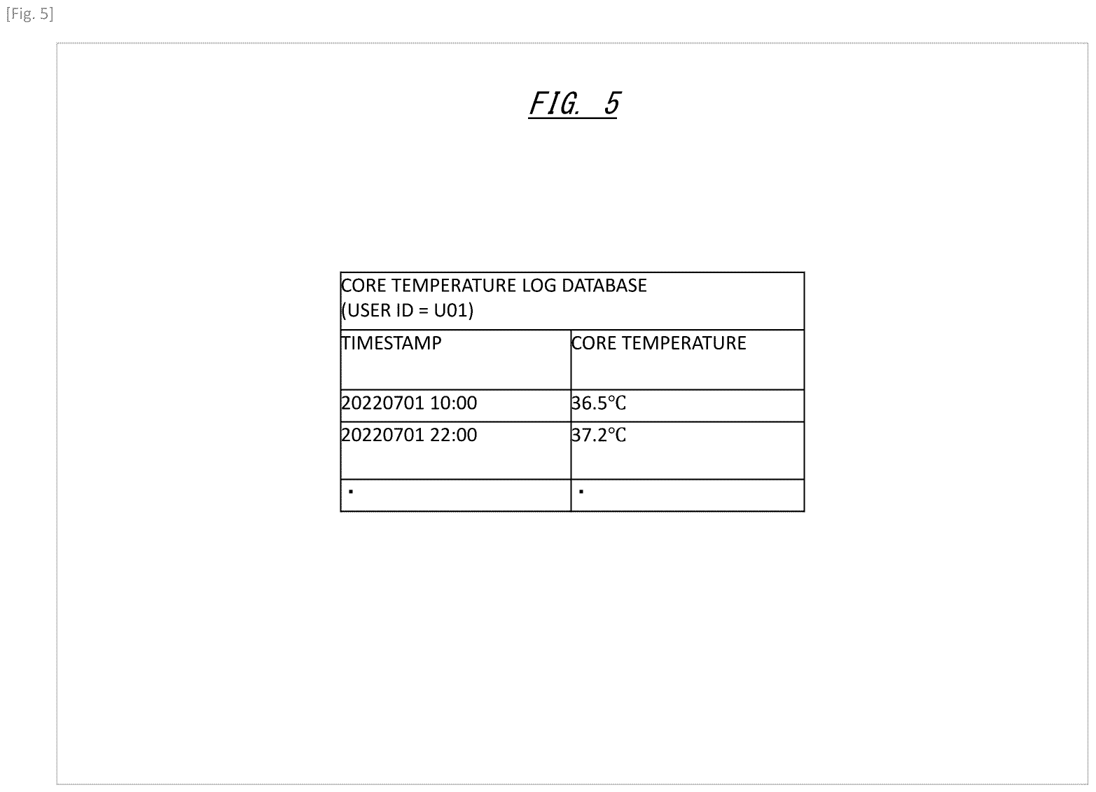

FIG. 5 is a diagram showing the data structure of the core temperature log database of the present embodiment.

The core temperature log database of FIG. 5 stores core temperature log information.

The core temperature log information is information on the user's core temperature history.

The core temperature history is at least one of the core temperature history measured periodically and the core temperature history measured irregularly.

The core temperature log database includes a “timestamp” field, and a “core temperature” field.

Each field is associated with each other.

The core temperature log database is associated with the user identification.

The “timestamp” field stores timestamp information.

The time stamp information is information on the date and time of the core temperature log.

The “core temperature” field stores core temperature information.

The core temperature information is information on the core temperature of the user.

The core temperature information may be obtained, for example, from at least one of the following:
Core temperature information inputted by the user;
Core temperature information measured by a core thermometer used by the user;
Core temperature information measured by a wearable device worn by the user; and
Infrared sensor capable of measuring core temperature.

### (3-3) Skin Care Log Database

The skin care log database of the present embodiment will be described.

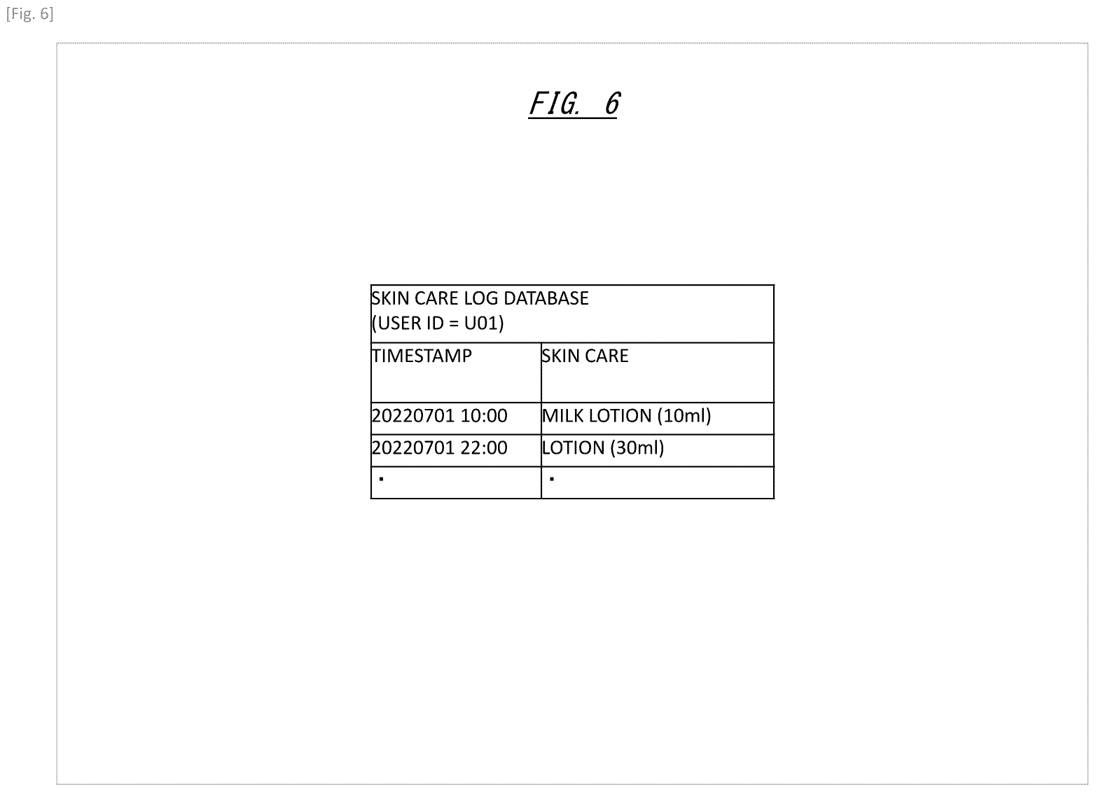

FIG. 6 is a diagram showing the data structure of the skin care log database of the present embodiment.

The skin care log database of FIG. 6 stores skin care log information.

The skin care log information is a history of skin care information.

The skin care information is information on skin care by the user.

The skin care log database includes a “timestamp” field, and a “skin care” field. Each field is associated with each other.

The skin care log database is associated with the user identification information.

The “timestamp” field stores timestamp information.

The time stamp information is information on the date and time of the skin care log.

The “skin care” field stores skin care information.

The skin care information is information on skin care.

The skin care information may include, for example, at least one of the following:
Ingredients in skin care products (for example, active ingredients, extract derivatives, or fragrance components);
Efficacy in skin care products (for example, moisturizing type or hydrating type);
Type of skin care products (for example, lotion, milky lotion, face mask, face pack, gel mask, oil serum, or all-in-one gel);
Used amount of skin care products;
Timing of using skin care products (for example, at the time of using facial care device);
Type of face massage at the time of skin care (for example, massage along nasolabial folds); and
Motion of body at the time of skin care (for example, stretching, yoga, or bathing).

From the skin care log information, the timing and frequency of skin care practice for each skin care content can be specified.

From the skin care log information, it can be specified, for example, that EXL Company's lotion is applied twice a day (for example, after washing the face in the morning and after taking a bath in the evening).

### (3-4) Physical Condition Log Database

The physical condition log database of the present embodiment will now be described.

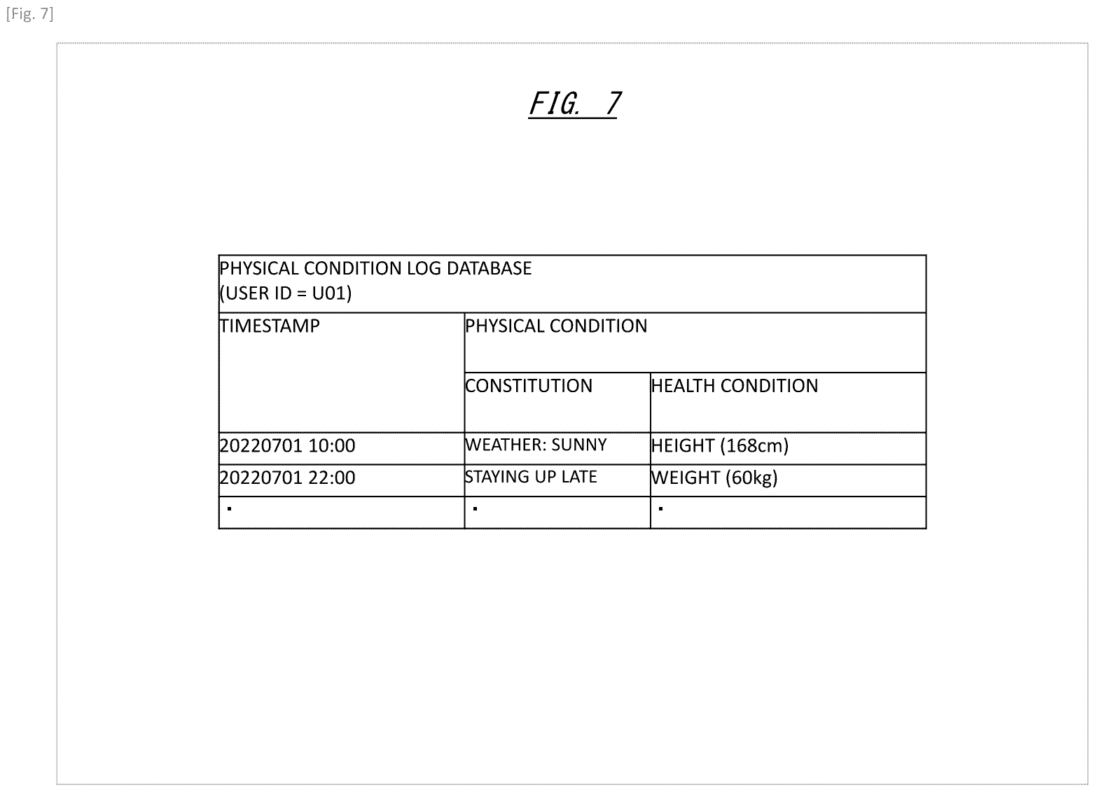

FIG. 7 is a diagram showing the data structure of the physical condition log database of the present embodiment.

The physical condition log database in FIG. 7 stores physical condition log information.

The physical condition log information is a history of the physical condition information.

The physical condition information is information on condition of the user's body (hereinafter referred to as “physical condition”).

The physical condition log database includes a “timestamp” field, and a “physical condition” field.

Each field is associated with each other.

The physical condition log database is associated with the user identification.

The “timestamp” field stores timestamp information.

The time stamp information is information on the date and time of the physical condition log.

The “physical condition” field stores physical condition information.

The “physical condition” field includes a “constitution” field and a “health condition” field.

The “Constitution” field stores constitution information.

The constitutional information is information on disruption of constitution (particularly constitutions that affect the manifestation of symptoms on the face).

The constitution information includes, for example, at least one of the following:
Disruption of constitution due to environmental factors (for example, weather, season, air pressure, ultraviolet rays, pollen, PM2.5, or midnight sun)
Disruption of constitution due to social factors (for example, stress, travel destinations (for example, country travel or jet lag), staying up late, or blue light (for example, smartphone use); and
Disruption of constitution due to physical activity (for example, disruption of growth hormone imbalance, disruption due to menstrual cycle, or disruption due to high-intensity exercise).

The “health condition” field stores health condition information.

The health condition information is information on the health condition (for example, information obtained from a medical checkup).

The health condition information includes, for example, at least one of the following:
Height;
Body weight;
Blood pressure;
Body fat mass;
Body fat percentage;
Waist circumference;
Blood sugar level;
BMI (Body Mass Index) value;
Urinalysis results (for example, uropyrinogen value, pH value, or specific gravity);
Blood test results (for example, AST, ALT, γ-GTP, hemoglobin level, red blood cell count, hematocrit level, white blood cell count (WBC), platelet count (PLT), or CRP (reactive protein));
Blood glucose level (for example, FPG or NGSP);
Lipid test results (for example, total cholesterol, HDL, LDL, or triglycerides); and
Renal function test results (for example, creatinine or uric acid).

### (3-5) Mental Condition Log Database

The mental condition log database of the present embodiment will now be described.

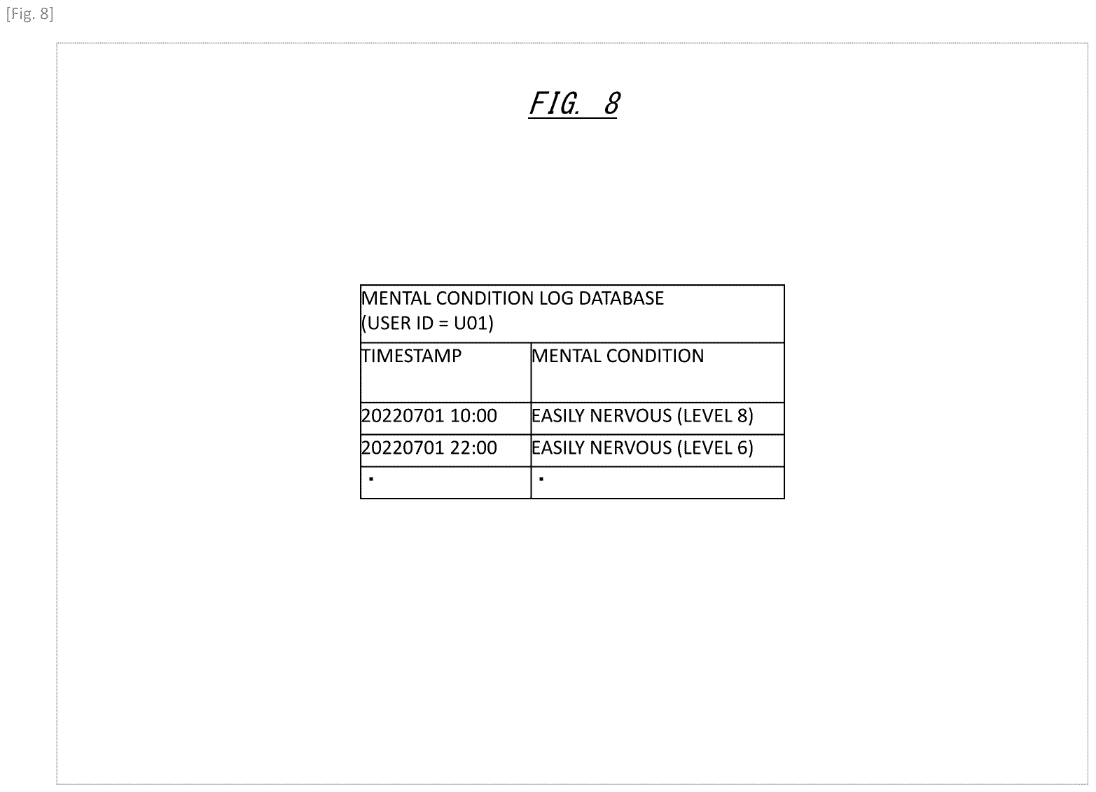

FIG. 8 is a diagram showing the data structure of the mental condition log database of the present embodiment.

The mental condition log database of FIG. 8 stores mental condition log information.

The mental condition log information is a history of the user's mental condition information.

The mental condition log database includes a “timestamp” field, and a “mental condition” field.

Each field is associated with each other.

The mental condition log database is associated with the user identification.

The “timestamp” field stores timestamp information.

The time stamp information is information on the date and time of the mental condition log.

The “mental condition” field stores mental condition information.

The mental condition information is information on the condition of the user's mental (hereinafter referred to as “mental condition”).

The mental condition information includes, for example, at least one of the following:
Information about a level of nervousness;
Information about stress state (for example, stress level specified from facial expression monitoring results);
Information about happiness state (for example, happiness level specified from facial expression monitoring results); and
Information about drowsiness state (for example, drowsiness level specified from the measurement results of a known sensor).

### (3-6) Human Organism Log Database

The organism log database of the present embodiment will be described.

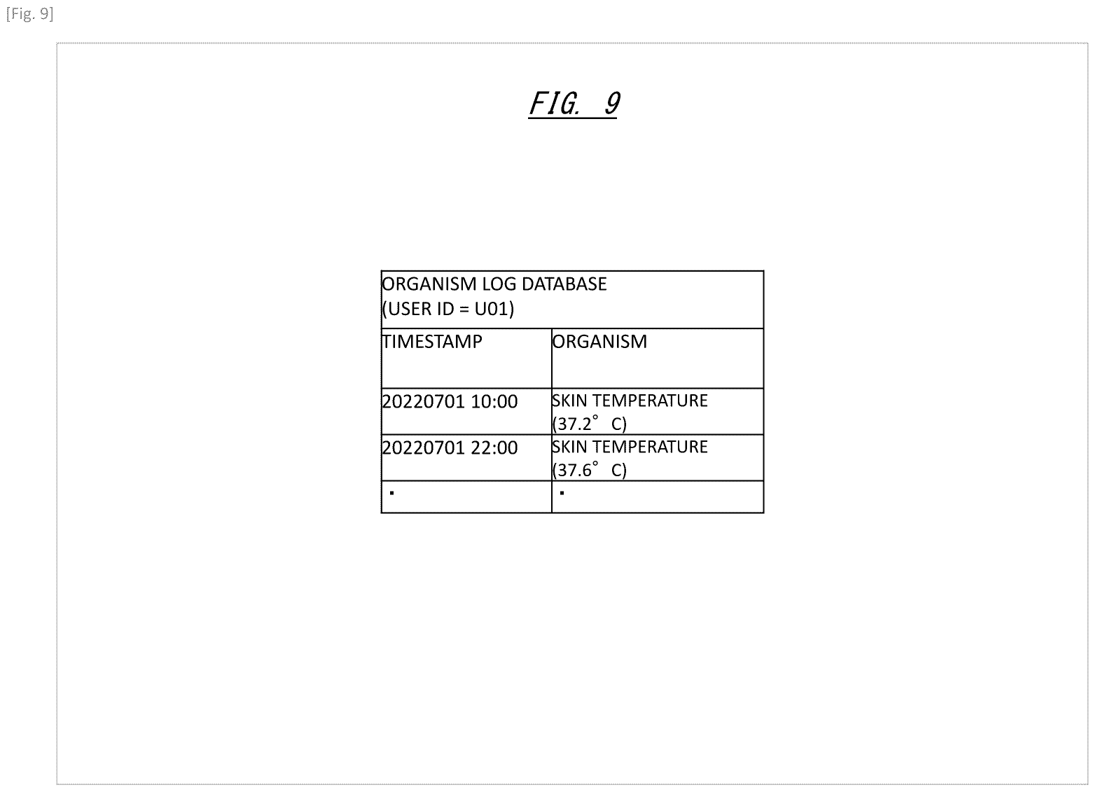

FIG. 9 is a diagram showing the data structure of the organism log database of the present embodiment.

The organism log database of FIG. 9 stores organism log information.

The organism log information is a history of the user's organism information. The organism log database includes a “timestamp” field and a “organism” field. Each field is associated with each other.

The organism log database is associated with the user identification.

The “timestamp” field stores timestamp information.

The time stamp information is information on the date and time of the organism log.

The “organism” field stores organism information.

Biometric information is information on the user's organism.

The organism information indicates, for example, at least one of the following:
Cutaneous temperature;
Skin temperature;
Environment temperature where the user spends time;
Pulse;
Heart rate;
Respiratory rate;
Electrocardiogram; and
Electromyography.

### (3-7) Action Log Database

The action log database of the present embodiment will be described.

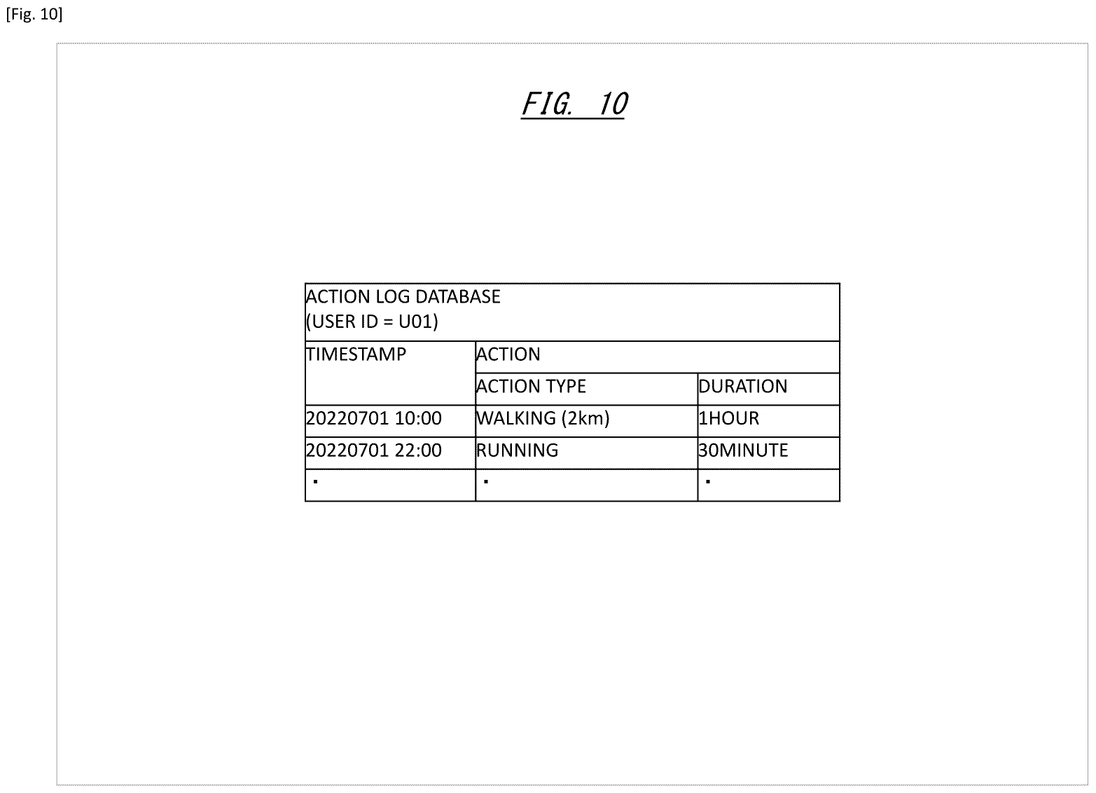

FIG. 10 is a diagram showing the data structure of the action log database of the present embodiment.

The action log database in FIG. 10 stores action log information.

The action log information is a history of the user's action information.

The action log database includes a “timestamp” field, and an “action” field.

Each field is associated with each other.

The action log database is associated with the user identification information.

The “timestamp” field stores timestamp information.

The time stamp information is information on the date and time of the action log.

The “action” field stores action information.

The action information is information on the user's action.

The “action”field includes an “action type” field and a “duration” field.

The “action type” field stores action type information.

The action type information is information on the type of action.

The type of activity includes the content of the activity and the amount of activity.

The type of action includes, for example, at least one of the following:
Lifestyle;
Rhythm of sleep;
Rhythm of meal/supplement (what time do you eat);
Rhythm of beauty (what time do you do your beauty action);
Habits of bathing and showering (what time do you take care of your beauty action);
Habits of light exercise; and
Exercise (for example, walking distance, running distance, number of steps, number of steps up and down the stairs, type of activity (for example, workout, exercise, or standing), number of pushes, moving distance, driving distance by bike or wheelchair).

The “duration” field stores duration information.

The duration information is information on the duration of the action.

From the action log information, it can be determined, for example, that a person performs stretching and yoga for 30 minutes at home between 8:00 and 9:00 on a weekday.

### (4) Information Processing

The information processing of the present embodiment will be described.

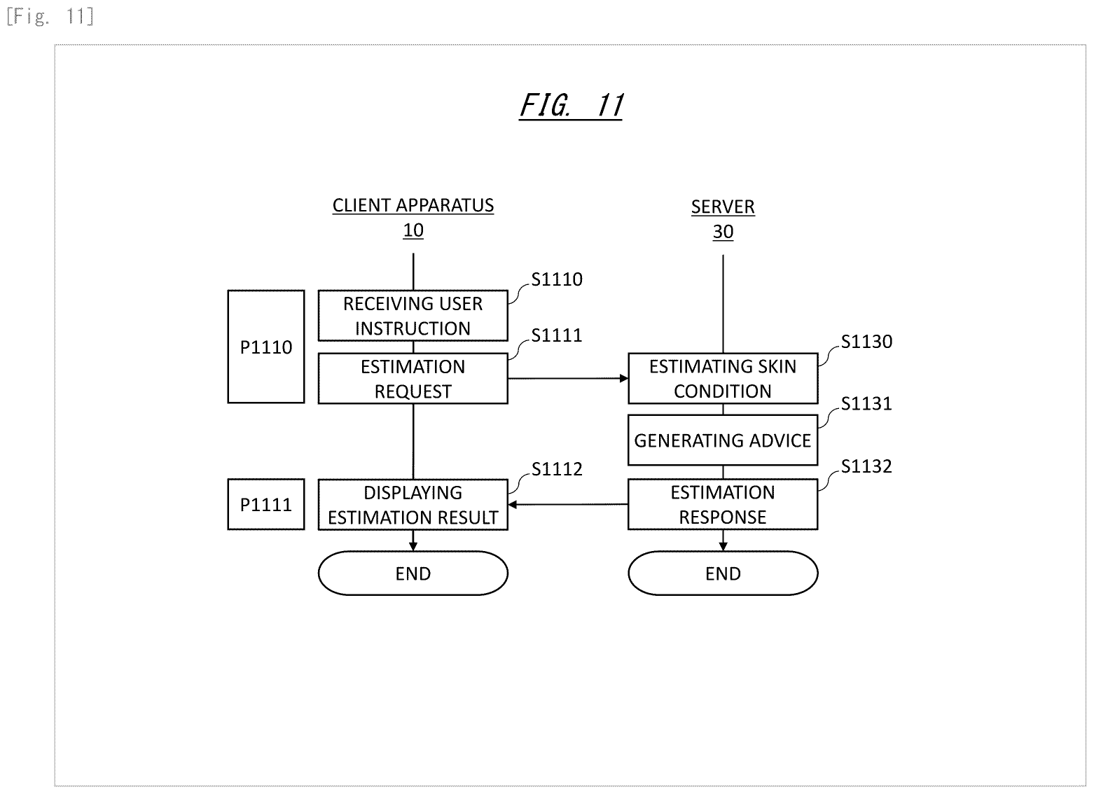

FIG. 11 is a sequence diagram of information processing of the present embodiment.

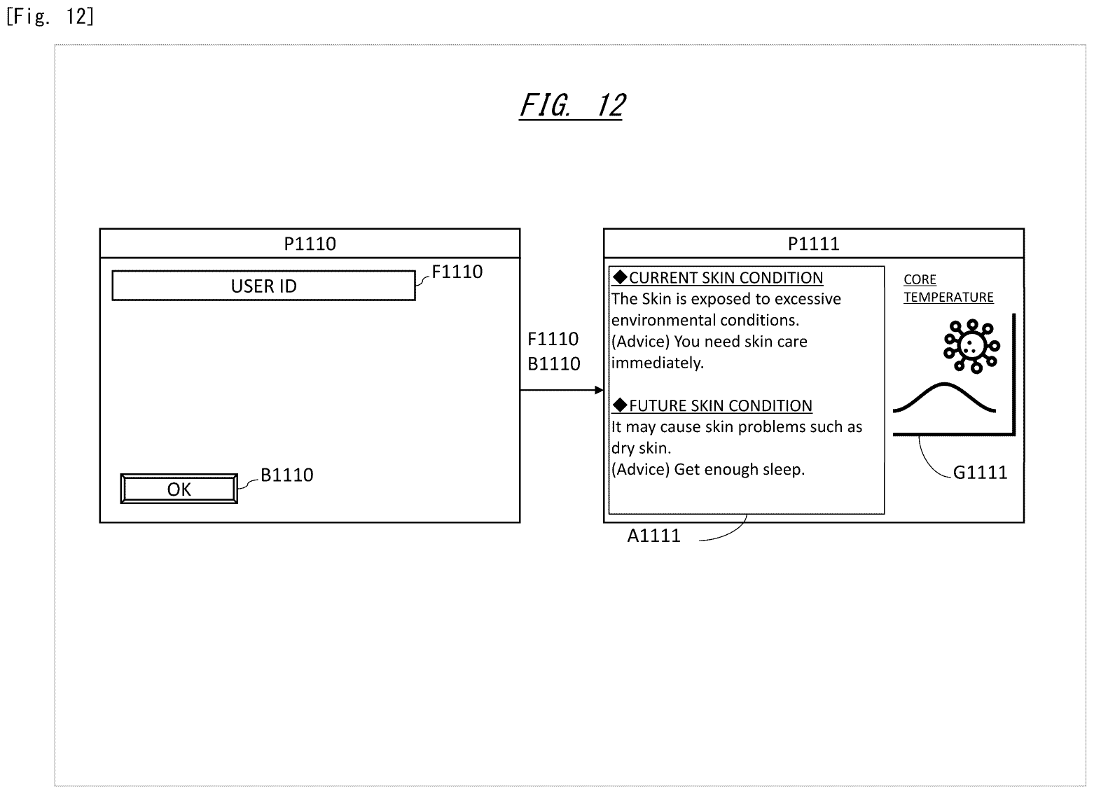

FIG. 12 is a diagram showing an example of a screen displayed in the information processing of FIG. 11.

The information processing in FIG. 8 is processing for estimating skin condition.

The information processing in FIG. 8 is triggered when a user accesses a predetermined website using the client apparatus 10.

As shown in FIG. 8, the client apparatus 10 executes receiving user instruction (S1110).

Specifically, the processor 12 displays screen P1110 (FIG. 12) on the display.

The screen P1110 includes an operation object B1110 and a field object F1110.

The operation object B1110 is an object that receives a user instruction for finalizing an input to the field object F1110.

The field object F1110 is an object that accepts the input of user identification information.

After step S1110, the client apparatus 10 executes estimation request (S1111). Specifically, when the user inputs user identification information into the field object F1110 and operates the operation object B1110, the processor 12 transmits estimation request data to the server 30.

The estimation request data includes, for example, the following information:
User identification information inputted in the field object F1110.

After step S1111, the server 30 executes estimating skin condition (S1130).

Specifically, the memory 31 stores the skin condition model.

The skin condition model describes the correlation between the core temperature history and skin condition.

The skin condition model includes at least one of the current skin condition model and the future skin condition model.

The skin condition may include, for example, at least one of the following:

The skin condition may include, for example, at least one of the following:
Physical condition (for example, skin viscoelasticity, stratum corneum moisture content, stratum corneum barrier function, antioxidant function, sebum volume, blood flow, stratum corneum condition, skin color, skin flexibility, glycation level, blood, urine, and sebum RNA); and
Qualitative condition (for example, skin age, skin moisture, skin sagging, skin condition, makeup application, and susceptibility to worsening of skin disorders (for example, acne or rough skin)).

As an example, the relationship between the physical state and the qualitative state is as follows:
When both viscoelasticity and moisture content of the stratum corneum decrease, skin age deteriorates;
When viscoelasticity increases, the moisture content of the stratum corneum increases, and the amount of sebum exceeds a certain level, the skin becomes less moisturized;
When viscoelasticity decreases, skin sagging worsens;
When viscoelasticity and the moisture content of the stratum corneum decrease, the amount of sebum exceeds a certain level, and the skin color becomes pale, the skin loses texture, firmness, and luster, resulting in a deterioration in the condition of the skin;
When the moisture content of the stratum corneum increases and the amount of sebum exceeds a certain level, makeup does not adhere well to the skin; and
When the moisture content of the stratum corneum decreases and the amount of sebum exceeds a certain level, the susceptibility of skin diseases to worsening increases.

The current skin condition model describes the correlation between the core temperature history and the current skin condition.

The current skin condition is the skin condition at the time when step S1130 is executed (hereinafter referred to as the “current time”).

The current skin condition model is configured to, when core temperature log information is input, output the current skin condition corresponding to the core temperature log information.

The future skin condition model describes the correlation between the core temperature history and the future skin condition.

The future skin condition is the skin condition at a time point in the future from the present time point.

The future skin condition model is configured to output the future skin condition corresponding to the core temperature log information when the core temperature log information is input.

The future time is a predetermined time.

The future time point is preferably two weeks from the current time point, taking into account the skin turnover cycle (for example, two weeks).

The future skin condition indicates, for example, a prediction of the tendency of changes in the skin condition that will occur in the future (for example, an improvement or deterioration of the skin condition).

The processor 32 refers to a core temperature log database (FIG. 5) associated with the user identification information included in the estimation request data, and specifies core temperature log information for a certain period (for example, one month prior to the date and time of execution of step S1130).

The processor 32 inputs the specified core temperature log information into the current skin condition model, and outputs the current skin condition corresponding to the core temperature log information.

The processor 32 inputs the specified core temperature log information into the future skin condition model, and outputs the future skin condition corresponding to the core temperature log information.

After step S1130, the server 30 executes generating advice (S1131).

Specifically, the memory 31 stores an advice model.

The advice model describes the correlation between the skin condition and the advice.

A first example of step S1131 will be described.

The advice model describes the correlation between the current skin condition and the advice information.

The advice information is information on advice according to the current skin condition.

The processor 32 inputs the current skin condition obtained in step S1130 into the advice model, and outputs advice information corresponding to the current skin condition.

The advice information indicates, for example, at least one of the following:
Advice on how to care for your skin; and
Advice on skin care products or makeup products which are recommended for use.

The advice according to the current skin condition preferably indicates at least one of the following:
Messages warning about skin risks (for example, the message “Your skin is exposed to excessive environmental condition”); and
Messages that encourage immediate action (for example, a message like “Your skin is exposed to excessive environmental condition and you need to take care of it immediately”).

A second example of Step S1131 will be described.

The advice model describes the correlation between the future skin condition and the advice information.

The advice information is information on advice according to the future skin condition.

The processor 32 inputs the future skin condition obtained in step S1130 into the advice model, and outputs advice information corresponding to the future skin condition.

The advice information indicates, for example, at least one of the following:
Advice on how to care for your skin; and
Advice on skin care products or makeup products which are recommended for use.

The advice according to the future skin condition preferably indicates at least one of the following:
Message warning you about skin problems (for example, message warning “Your skin may be in bad condition due to a woman's cycle” or “Your sleep rhythm was poor last night, so you may be experiencing skin problems such as rough skin”); and
Message encouraging action to improve skin problems (for example, message encouraging “You may develop skin problems such as rough skin, so please get enough sleep”).

The first and second examples of step S1131 can be combined.

After step S1131, the server 30 executes estimation response (S1132).

Specifically, the processor 32 transmits the estimation response data to the client apparatus 10.

The estimation response data includes, for example, the following information:
Information about the current skin condition obtained in step S1130 (hereinafter referred to as “current skin condition information”);
Information about the future skin condition obtained in step S1130 (hereinafter referred to as “future skin condition information”);
Advice information obtained in step S1131; and
Core temperature log information used in step S1130.

After step S1132, the client apparatus 10 displays estimation result (S1112).

Specifically, the processor 12 displays screen P1111 (FIG. 12) on the display.

The screen P1111 includes a display object A1111 and a graph object G1111.

The display object A1111 displays the current skin condition information, the future skin condition information, and the advice information contained in the estimation response data.

The graph object G1111 is a graph showing core temperature log information (that is, time-series changes in core temperature) included in the estimation response data.

### (5) Summary of the Present Embodiment

According to the present embodiment, the skin condition is estimated based on the core temperature history.

This makes it possible to improve the accuracy of estimating skin condition compared to the conventional method.

For example, in the present embodiment, the skin condition is estimated based on the core temperature (that is, higher-dimensional sensing data for the skin) obtained by a device that is directly attached to the skin (that is, a wearable device).

The core temperature includes factors that are not yet occurred at the skin surface.

This makes it possible to estimate skin condition taking into account factors not taken into account in conventional skin condition estimations (for example, prediction of cell turnover, movement of macrophages in the dermis, or immune pathways).

According to the present embodiment, the user's future skin condition may be estimated based on the core temperature history.

This allows the user to know accurately his/her future skin condition.

According to the present embodiment, the future skin condition may be a tendency of changes in the skin condition that will occur to the skin in the future.

This allows the user to know future changes in the condition of their skin.

According to the present embodiment, the user's current skin condition may be estimated based on the core temperature history.

This allows the user to accurately know their current skin condition.

According to the present embodiment, advice according to the estimation result of the skin condition may be presented to the user.

This allows the user to take action (for example, skin care action) based on advice that corresponds to the accurate skin condition.

### (6) Modification

A modification of the present embodiment will now be described.

### (6-1) First Modification

The first modification will be described.

The first modification is an example in which at least one of the current skin condition and the future skin condition is estimated based on the core temperature history and a skin care history.

### (6-1-1) Overview of First Modification

The overview of the first modification will be described.

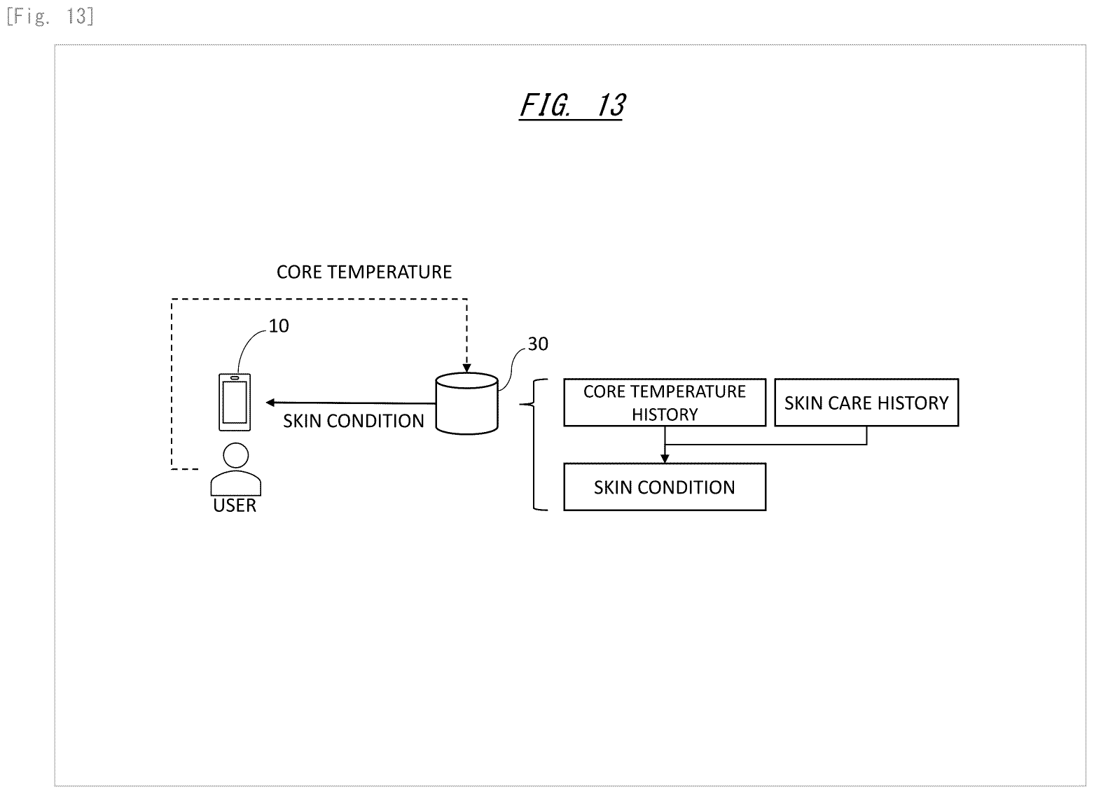

FIG. 13 is a diagram illustrating an overview of the first modification.

As shown in FIG. 13, the server 30 stores the user's core temperature history and skin care history.

The server 30 estimates the user's skin condition based on the core temperature history and the skin care history.

The server 30 presents the estimation result (that is, the user's skin condition) to the user via the client apparatus 10.

### (6-1-1) Information Processing of First Modification

The information processing of the first modification will be described.

As shown in FIG. 11, the client apparatus 10 executes the steps from receiving user instruction (S1110) to estimation request (S1111) in the same manner as in the present embodiment.

After step S1111, the server 30 executes estimating skin condition (S1130).

Specifically, the memory 31 stores the skin condition model.

The skin condition model describes the correlation between the core temperature history, the skin care history, and the skin condition.

The skin condition model includes at least one of the current skin condition model and the future skin condition model.

The current skin condition model describes the correlation between the core temperature history and the skin care history, and the current skin condition.

The future skin condition model describes the correlation between the core temperature history and the skin care history, and the future skin condition.

The processor 32 refers to the core temperature log database (FIG. 5) associated with the user identification information included in the estimation request data to specify the core temperature log information.

The processor 32 refers to the skin care log database (FIG. 6) associated with the user identification information included in the estimation request data to specify the skin care history.

The skin care history is specified by skin care information for a predetermined period (for example, one month prior to the present time) or the most recent skin care information.

The processor 32 inputs the specified core temperature log information and skin care information into the current skin condition model, and outputs the current skin condition corresponding to the core temperature log information and the skin care information.

The processor 32 inputs the specified core temperature log information and the skin care information into the future skin condition model to output the future skin condition corresponding to the core temperature log information and the skin care information.

After step S1130, the server 30 executes the steps from generating advice (S1131) to estimation response (S1132) in the same manner as in the present embodiment.

After step S1132, the client apparatus 10 executes displaying estimation result (S1112) in the same manner as in the present embodiment.

### (6-1-3) Summary of First Modification

According to the first modification, the skin condition may be estimated based on the core temperature history and the skin care history.

This makes it possible to further improve the accuracy of estimating the skin condition, and also makes it possible to present advice that is more suitable for improving the skin condition.

### (6-2) Second Modification

The second modification will now be described.

The second modification is an example in which the skin condition is estimated based on the core temperature history and a physical condition history.

### (6-2-1) Overview of Second Modification

The overview of the second modification will now be described.

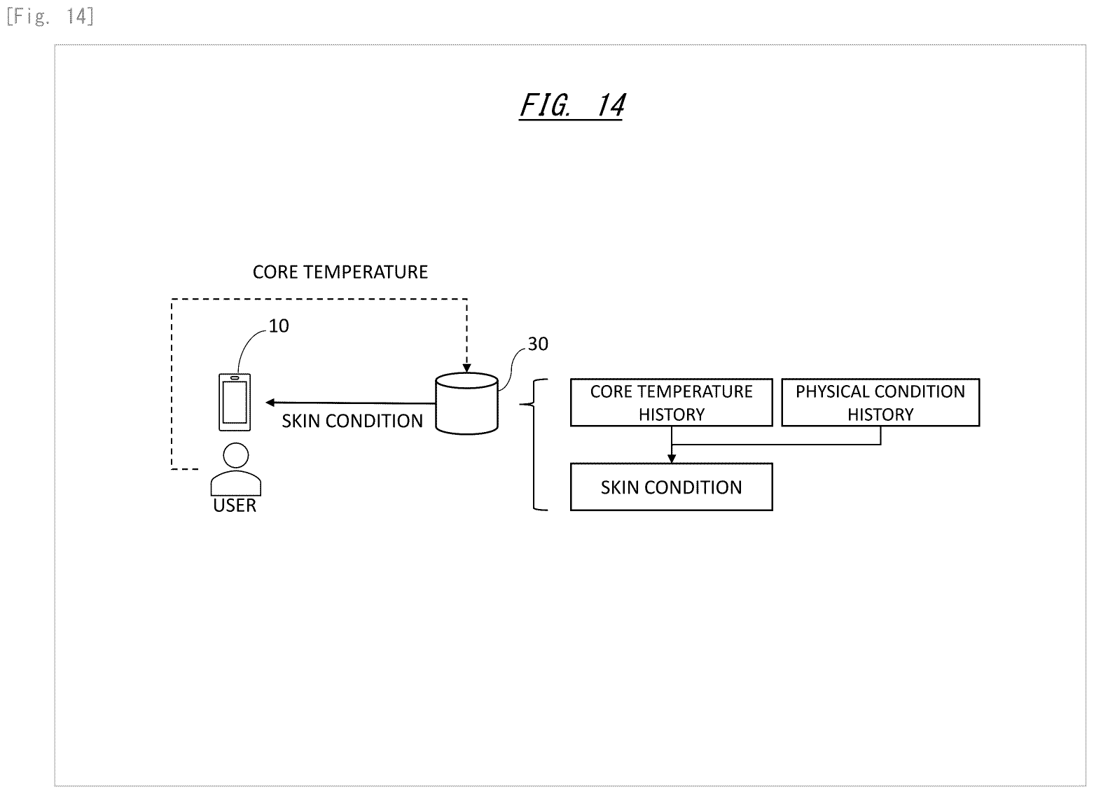

FIG. 14 is a diagram illustrating an overview of the second modification.

As shown in FIG. 154, the server 30 stores the user's core temperature history and the physical condition history.

The server 30 estimates the user's skin condition based on the core temperature history and the physical condition history.

The server 30 presents the estimation result (that is, the user's skin condition) to the user via the client apparatus 10.

### (6-2-2) Information Processing of Second Modification

The information processing of the second modification will be described.

As shown in FIG. 11, the client apparatus 10 executes the steps from receiving user instruction (S1110) to estimation request (S1111) in the same manner as in the present embodiment.

After step S1111, the server 30 executes estimating skin condition (S1130).

Specifically, the memory 31 stores the skin condition model.

The skin condition model describes the correlation between the core temperature history and the physical condition history, and the skin condition.

The skin condition model includes at least one of the current skin condition model and the future skin condition model.

The current skin condition model describes the correlation between the core temperature history, the physical condition history, and the current skin condition.

The future skin condition model describes the correlation between the core temperature history, the physical condition history, and the future skin condition.

The processor 32 refers to the core temperature log database (FIG. 5) associated with the user identification information included in the estimation request data to specify the core temperature log information.

The processor 32 refers to the physical condition log database (FIG. 7) associated with the user identification information included in the estimation request data to specify the physical condition history.

The physical condition history is specified by the physical condition information for a predetermined period (for example, one month prior to the present time) or the most recent physical condition information.

The processor 32 inputs the specified core temperature log information and physical condition information into the current skin condition model to output the current skin condition corresponding to the core temperature log information and the physical condition information.

The processor 32 inputs the specified core temperature log information and physical condition information into the future skin condition model to output the future skin condition corresponding to the core temperature log information and the physical condition information.

After step S1130, the server 30 executes the steps from generating advice (S1131) to estimation response (S1132) in the same manner as in the present embodiment.

After step S1132, the client apparatus 10 executes displaying estimation result (S1112) in the same manner as in the present embodiment.

### (6-2-3) Summary of Second Modification

According to the second modification, the skin condition may be estimated based on the core temperature history and the physical condition history.

This makes it possible to further improve the accuracy of estimating the skin condition, and also makes it possible to present advice that is more suitable for improving the skin condition.

### (6-3) Third Modification

The third modification will now be described.

The third modification is an example in which the skin condition is estimated based on the core temperature history and a mental condition history.

### (6-3-1) Overview of Third Modification

The overview of the third modification will now be described.

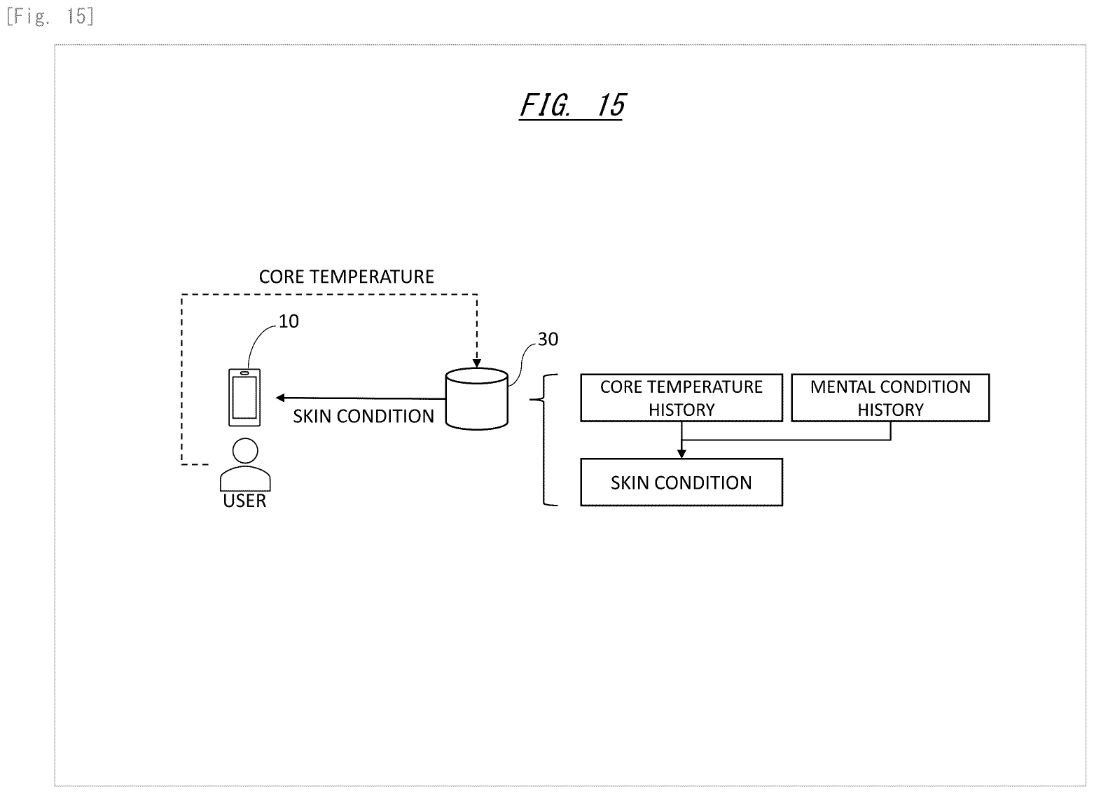

FIG. 15 is a diagram illustrating an overview of the third modification.

As shown in FIG. 15, the server 30 stores the user's core temperature history and the mental condition history.

The server 30 estimates the user's skin condition based on the core temperature history and the mental condition history.

The server 30 presents the estimation result (that is, the user's skin condition) to the user via the client apparatus 10.

### (6-3-2) Information Processing of Third Modification

The information processing of the third modification will be described.

As shown in FIG. 11, the client apparatus 10 executes the steps from receiving user instruction (S1110) to estimation request (S1111) in the same manner as in the present embodiment.

After step S1111, the server 30 executes estimating skin condition (S1130).

Specifically, the memory 31 stores the skin condition model.

The skin condition model describes the correlation between the core temperature history, the mental condition history, and the skin condition.

The skin condition model includes at least one of the current skin condition model and the future skin condition model.

The current skin condition model describes the correlation between the core temperature history, the mental condition history, and the current skin condition.

The future skin condition model describes the correlation between the core temperature history, the mental condition history, and the future skin condition.

The processor 32 refers to the core temperature log database (FIG. 5) associated with the user identification information included in the estimation request data to specify the core temperature log information.

The processor 32 refers to the mental condition log database (FIG. 8) associated with the user identification information included in the estimation request data to specify the mental condition history.

The mental condition history is specified by mental condition information for a predetermined period (for example, one month prior to the present time) or the most recent mental condition information.

The processor 32 inputs the specify core temperature log information and the mental condition information into the current skin condition model to output the current skin condition corresponding to the core temperature log information and the mental condition information.

The processor 32 inputs the specify core temperature log information and the mental condition information into the future skin condition model to output the future skin condition corresponding to the core temperature log information and the mental condition information.

After step S1130, the server 30 executes the steps from generating advice (S1131) to estimation response (S1132) in the same manner as in the present embodiment.

After step S1132, the client apparatus 10 executes displaying the estimation result (S1112) in the same manner as in the present embodiment.

### (6-3-3) Third Modification

According to the third modification, the skin condition may be estimated based on the core temperature history and the mental condition history.

This makes it possible to further improve the accuracy of estimating the skin condition, and also makes it possible to present advice that is more suitable for improving the skin condition.

### (6-4) Fourth Modification

The fourth modification will now be described.

The fourth modification is an example in which the skin condition is estimated based on the core temperature history and a history of organism logical information.

### (6-4-1) Overview of Fourth Modification

The overview of the fourth modification will now be described.

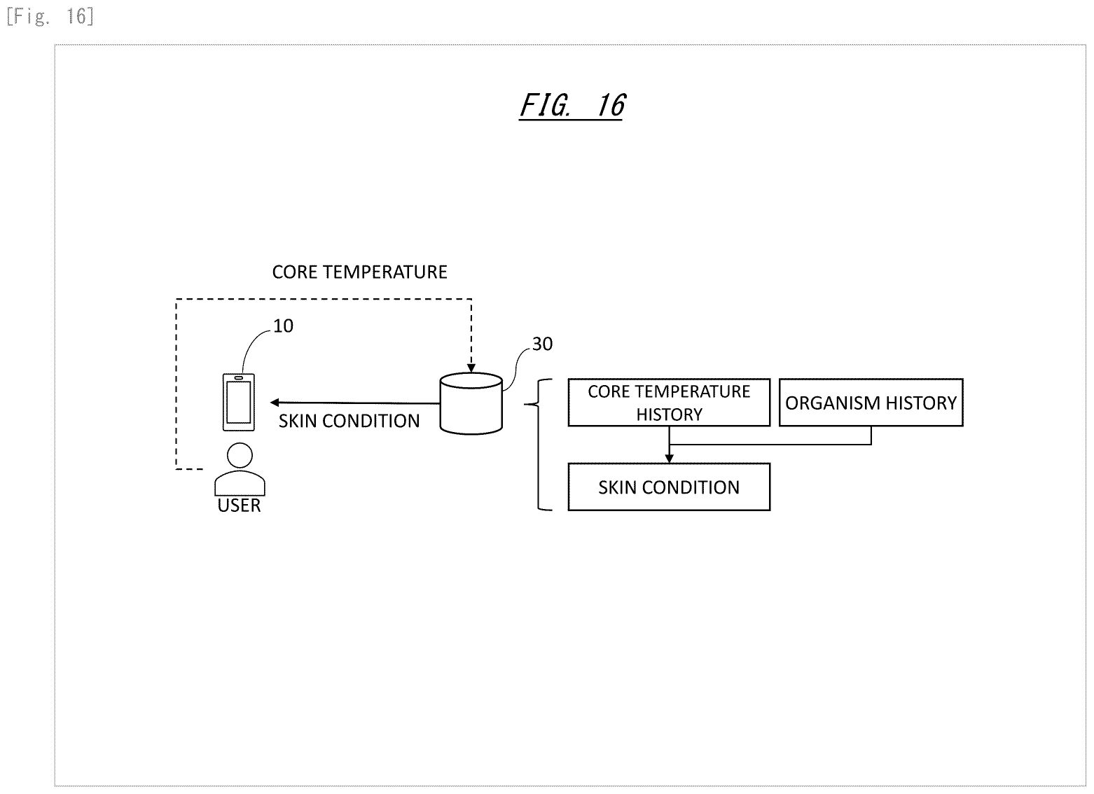

FIG. 16 is a diagram illustrating an overview of the fourth modification.

As shown in FIG. 16, the server 30 stores the user's core temperature history and the organism history.

The server 30 estimates the user's skin condition based on the core temperature history and the organism history.

The server 30 presents the estimation result (that is, the user's skin condition) to the user via the client apparatus 10.

### (6-4-2) Information Processing of Fourth Modification

The information processing of the fourth modification will be described.

As shown in FIG. 11, the client apparatus 10 executes the steps from receiving user instruction (S1110) to estimation request (S1111) in the same manner as in the present embodiment.

After step S1111, the server 30 executes estimating skin condition (S1130).

Specifically, the memory 31 stores the skin condition model.

The skin condition model describes the correlation between the core temperature history, the organism history, and the skin condition.

The skin condition model includes at least one of the current skin condition model and the future skin condition model.

The current skin condition model describes the correlation between the core temperature history, the organism history, and the current skin condition.

The future skin condition model describes the correlation between the core temperature history, the organism history, and the future skin condition.

The processor 32 refers to the core temperature log database (FIG. 5) associated with the user identification information included in the estimation request data to specify the core temperature log information.

The processor 32 refers to the organism log database (FIG. 9) associated with the user identification information included in the estimation request data to specify the organism history.

The organism history is specified by organism information for a predetermined period (for example, one month prior to the present time) or the most recent organism information.

The processor 32 inputs the specified core temperature log information and organism information into the current skin condition model to output the current skin condition corresponding to the core temperature log information and the organism information.

The processor 32 inputs the specified core temperature log information and the organism information into the future skin condition model to output the future skin condition corresponding to the core temperature log information and the organism information.

After step S1130, the server 30 executes the steps from generating advice (S1131) to estimation response (S1132) in the same manner as in the present embodiment.

After step S1132, the client apparatus 10 executes displaying estimation result (S1112) in the same manner as in the present embodiment.

### (6-4-3) Fourth Modification

According to the fourth modification, the skin condition may be estimated based on the core temperature history and the organism history.

This makes it possible to further improve the accuracy of estimating the skin condition, and also makes it possible to present advice that is more suitable for improving the skin condition.

### (6-5) Fifth Modification

The fifth modification will now be described.

The fifth modification is an example in which the skin condition is estimated based on the core temperature history and an action history.

### (6-5-1) Overview of Fifth Modification

The overview of the fifth modification will now be described.

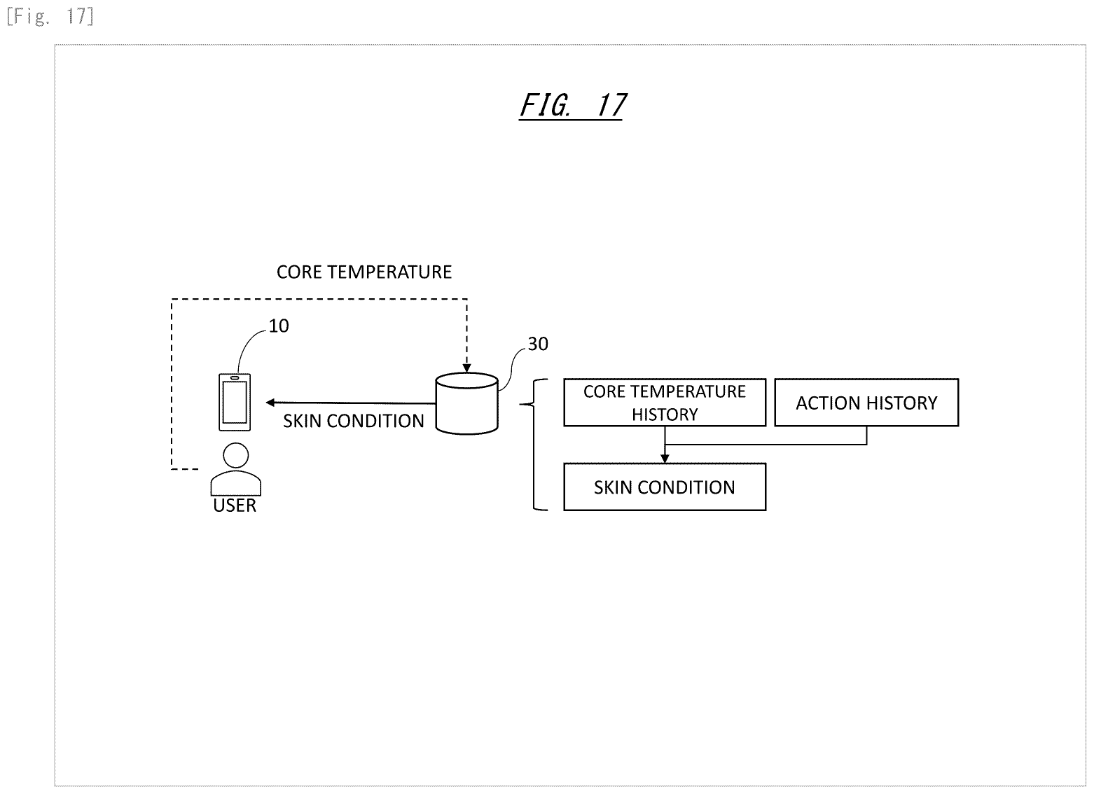

FIG. 17 is a diagram illustrating an overview of the fifth modification.

As shown in FIG. 17, the server 30 stores the user's core temperature history and the action history.

The server 30 estimates the user's skin condition based on the core temperature history and the action history.

The server 30 presents the estimation result (that is, the user's skin condition) to the user via the client apparatus 10.

### (6-5-2) Information Processing of Fifth Modification

The information processing of the fifth modification will be described.

As shown in FIG. 11, the client apparatus 10 executes the steps from receiving user instruction (S1110) to estimation request (S1111) in the same manner as in the present embodiment.

After step S1111, the server 30 executes estimating skin condition (S1130).

Specifically, the memory 31 stores the skin condition model.

The skin condition model describes the correlation between the core temperature history, the action history, and the skin condition.

The skin condition model includes at least one of the current skin condition model and the future skin condition model.

The current skin condition model describes the correlation between the core temperature history, the action history, and the current skin condition.

The future skin condition model describes the correlation between the core temperature history, the action history and the future skin condition.

The processor 32 refers to the core temperature log database (FIG. 5) associated with the user identification information included in the estimation request data to specify the core temperature log information.

The processor 32 refers to the action log database (FIG. 10) associated with the user identification information included in the estimation request data to specify the action history.

The action history is specified by the action information for a predetermined period (for example, one month prior to the present time) or the most recent action information.

The processor 32 inputs the specified core temperature log information and the action information into the current skin condition model to output the current skin condition corresponding to the core temperature log information and the action information.

The processor 32 inputs the specified core temperature log information and the action information into the future skin condition model to output the future skin condition corresponding to the core temperature log information and the action information.

After step S1130, the server 30 executes the steps from generating advice (S1131) to estimation response (S1132) in the same manner as in the present embodiment.

After step S1132, the client apparatus 10 executes displaying estimation result (S1112) in the same manner as in the present embodiment.

### (6-5-3) Summary of Fifth Modification

According to the fifth modification, the skin condition may be estimated based on the core temperature history and the action history.

This makes it possible to further improve the accuracy of estimating the skin condition, and also makes it possible to present advice that is more suitable for improving the skin condition.

The fifth modification can also be applied to an example in which the skin condition is estimated based on the core temperature history and a user's action plan.

For example, the memory 31 stores action plan information.

The action plan information is information on the user's future action plan.

The action plan information is associated with the user identification information.

### The Memory 31 Stores the Skin Condition Model.

The skin condition model describes the correlation between the core temperature history, the action plan, and the future skin condition.

In estimating the skin condition (S1130), the server 30 refers to the core temperature log database (FIG. 5) associated with the user identification information included in the estimation request data to specify the core temperature log information.

The processor 32 refers to the action plan information associated with the user identification information included in the estimation request data to specify the user's action plan.

The action plan is specified by the action plan information for a predetermined period (for example, one month from the present time into the future) or the action plan information for the immediately following period.

The processor 32 inputs the specified core temperature log information and the action plan information into the skin condition model to output the future skin condition corresponding to the core temperature log information and action plan information.

According to this example, the skin condition may be estimated based on the core temperature history and the action plan.

This makes it possible to further improve the accuracy of estimating the skin condition, and also makes it possible to present advice that is more suitable for improving the skin condition.

### (6-6) Sixth Modification

The sixth modification will now be described.

The sixth modification is an example in which advice is presented according to the user's preferences.

### (6-6-1) Overview of Sixth Modification

The overview of the sixth modification will now be described.

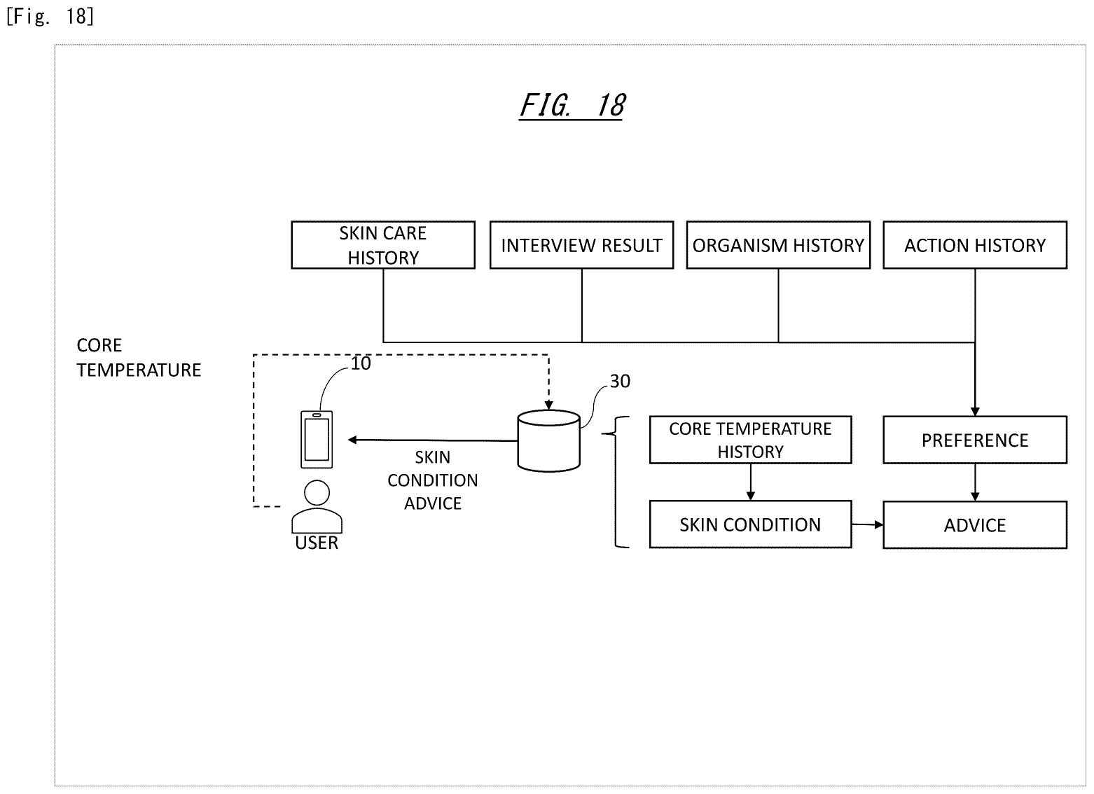

FIG. 18 is a diagram illustrating an overview of the sixth modification.

As shown in FIG. 18, the server 30 stores the user's core temperature history and the action history.

The server 30 estimates the user's skin condition based on the core temperature history.

The server 30 estimates the user's preferences based on at least one of the action history, the organism history, the results of the medical interview, and the skin care history.

The server 30 generates advice based on the user's skin condition and the user's preferences.

The server 30 presents the estimation result (that is, the user's skin condition) and advice to the user via the client apparatus 10.

### (6-6-2) Information Processing of Sixth Modification

The information processing of the sixth modification will be described.

As shown in FIG. 11, the client apparatus 10 executes the steps from receiving user instruction (S1110) to estimation request (S1111) in the same manner as in the present embodiment.

After step S1111, the server 30 executes estimating skin condition (S1130) in the same manner as in the present embodiment.

After step S1130, the server 30 executes generating advice (S1131).

Specifically, the memory 31 stores an advice model.

The advice model describes the correlation between the skin condition, the user's preference, and the advice.

A first example of step S1131 will be described.

The processor 32 refers to the action log database (FIG. 10) associated with the user identification information included in the estimation request data, and estimates the user's preferred action, such as action that the user is good at (for example, action that the user often performs).

The processor 32 inputs the skin condition and the preferred action into the advice model to output advice information that encourages the user to perform the preferred action according to the skin condition.

A second example of step S1131 will be described.

The processor 32 refers to the action log database (FIG. 10) associated with the user identification information included in the estimation request data to estimate unpleasant action that the user is not good at (for example, action of a duration shorter than the standard duration or action that occur less frequently than the standard frequency) as the user's preferred action.

The processor 32 inputs the skin condition and the unpleasant action into the advice model, and outputs advice information that encourages the user to perform action other than the unpleasant action according to the skin condition.

A third example of step S1131 will be described.

The processor 32 refers to the organism log database (FIG. 9) and the action log database (FIG. 10) associated with the user identification information included in the estimation request data to estimate the user's action preferences, such as preferences for action that produce significant bioreactions (for example, action that excite the user). The processor 32 inputs the skin condition and the estimation result into the advice model to output advice information that encourages the user to take an action that produces a significant organism response, according to the skin condition.

A fourth example of step S1131 will be described.

The memory 31 stores interview information.

The interview information is information on the results of a questionnaire administered to the user.

The interview information is associated with the user identification information.

The processor 32 refers to the interview information associated with the user identification information included in the estimation request data to estimate the user's preferences (for example, likes and dislikes).

The processor 32 inputs the skin condition and the estimation result into the advice model to output advice information suited to the user's preferences according to the skin condition.

A fifth example of Step S1131 will be described.

The processor 32 refers to the skin care log database (FIG. 6) associated with the user identification information included in the estimation request data to estimate the user's cosmetic preferences (for example, frequently used cosmetics or cosmetics that the user prefers).

The processor 32 inputs the skin condition and the estimation result into the advice model to output advice information suited to the user's preferences according to the skin condition.

The first to fifth examples of Step S1131 can be combined.

After step S1131, the server 30 executes estimation response (S1132) in the same manner as in the present embodiment.

After step S1132, the client apparatus 10 executes displaying estimation result (S1112) in the same manner as in the present embodiment.

### (6-6-3) Summary of Sixth Modification

According to the sixth modification, advice may be generated by referring to the skin condition estimation result and the user's action log information.

This makes it possible to provide advice that takes into account not only the user's skin condition but also the user's preferences of action.

According to the sixth modification, advice may be generated that encourages the user to perform actions that he or she is good at.

This makes it possible to encourage users to take actions that are easy to implement.

According to the sixth modification, advice may be generated that encourages the user to perform action other than the action that the user is not good at.

This makes it possible to encourage users to take actions that are easy to implement.

According to the sixth modification, advice may be generated that takes into consideration actions that produce significant organism reactions, based on a combination of the organism history and the action history.

This makes it possible to provide advice that takes into account not only the user's skin condition but also the user's preferences of action.

According to the sixth modification, the user's preferences may be estimated based on the results of the interview, and advice may be generated according to the estimated preferences.

This makes it possible to provide advice that takes into account not only the user's skin condition but also the user's preferences.

According to the sixth modification, the user's cosmetic preferences may be estimated based on the skin care history, and advice may be generated according to the estimated preferences.

This makes it possible to provide advice that takes into account not only the user's skin condition but also the user's cosmetic preferences.

### (6-7) Seventh Modification

The seventh modification will now be described.

The seventh modification is an example in which the skin condition is estimated based on a DPG (Distal Proximal-temperature Gradient) parameter history.

### (6-7-1) Overview of Seventh Modification

The overview of the seventh modification will now be described.

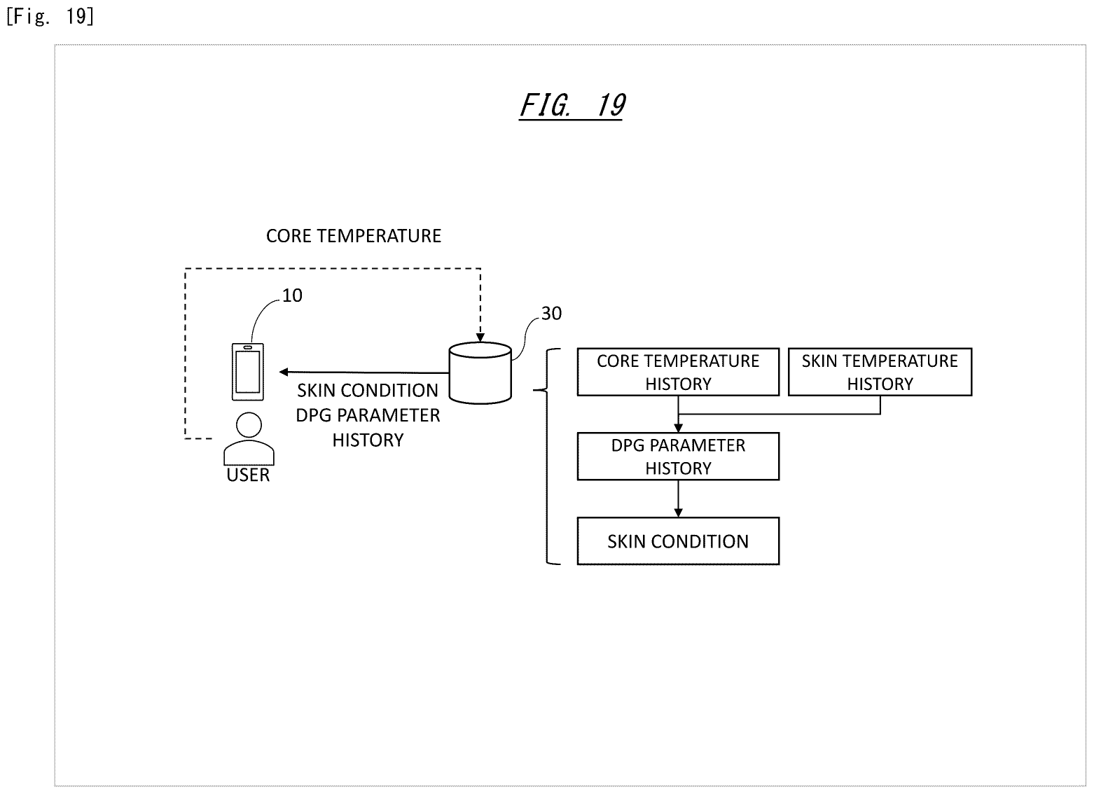

FIG. 19 is a diagram illustrating an overview of the seventh modification.

As shown in FIG. 19, the server 30 stores the user's core temperature history and the skin temperature history.

The server 30 calculates the DPG parameter history based on the core temperature history and the skin temperature history.

The server 30 estimates the user's skin condition based on the DPG parameter history.

The server 30 presents the estimation results (that is, the user's skin condition) and the DPG parameter history to the user via the client apparatus 10.

The DPG parameter is also referred to as a distal-proximal temperature gradient.

The DPG parameters are any of the following:
Difference between core temperature in the central part of the body and peripheral body temperature in extremities (for example, hands and feet);
Difference between peripheral skin temperature and the core temperature; and
Difference between distal body temperature and proximal body temperature;

It is generally known that sleep onset is promoted when the DPG is low, and the DPG parameters have similar characteristics.

### (6-7-2) Information Processing of Seventh Modification

The information processing of the seventh modification will be described.

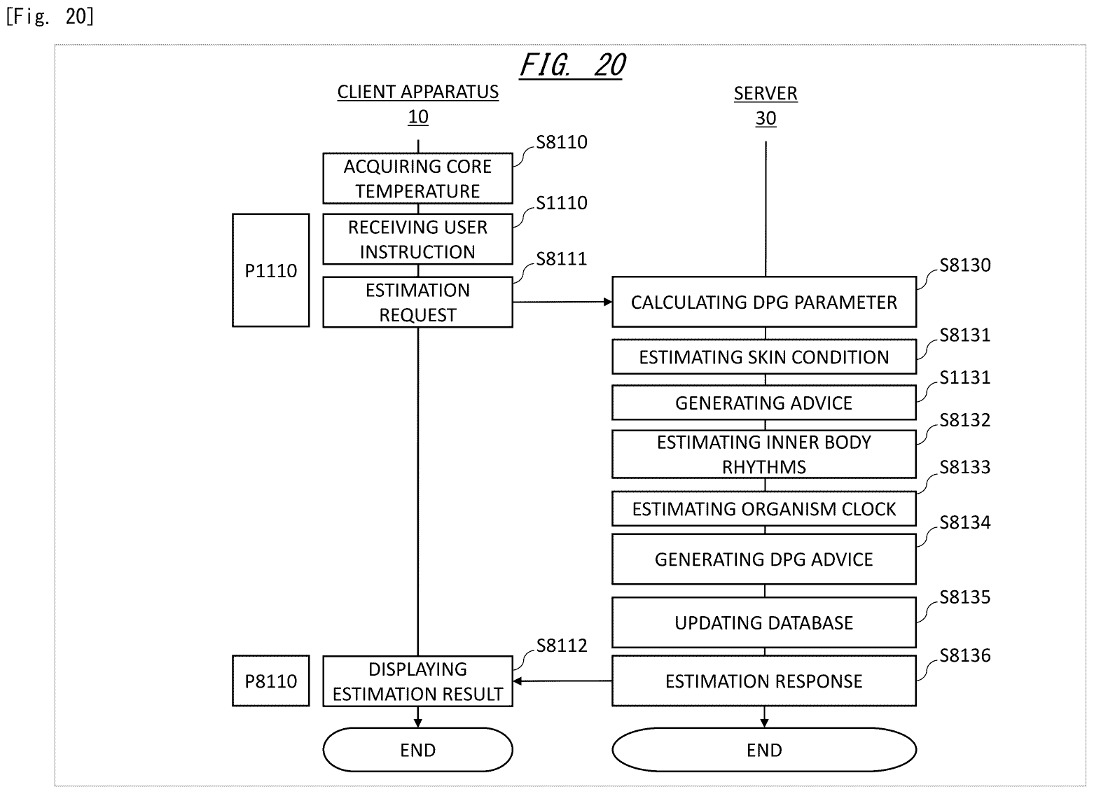

FIG. 20 is a sequence diagram of information processing of the seventh modification.

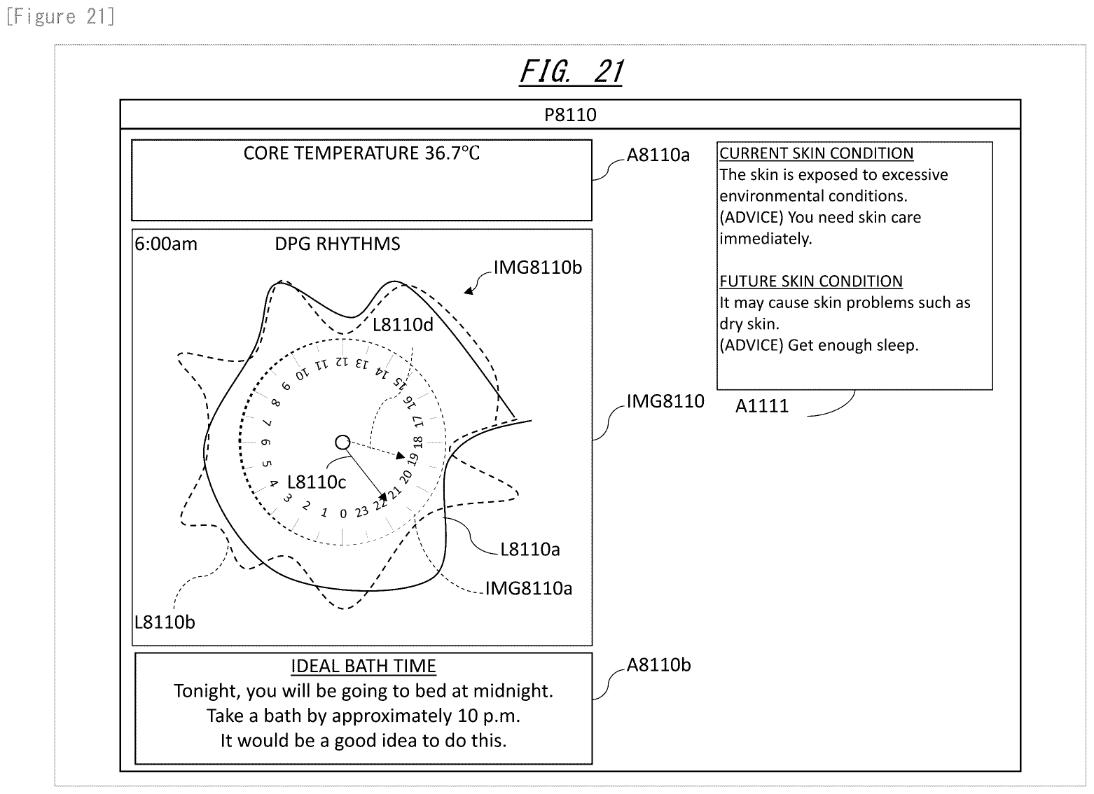

FIG. 21 is a view showing an example of a screen displayed in the information processing of FIG. 20.

As shown in FIG. 20, the client apparatus 10 executes acquiring core temperature (S8110).

Specifically, the processor 12 acquires the user's core temperature information. The core temperature information may be obtained, for example, from at least one of the following:
Core temperature information obtained from a core body thermometer used by the user;
Core temperature information obtained from a wearable device worn by the user; and
Infrared sensor capable of measuring core temperature.

After step S8110, the client apparatus 10 executes receiving user instruction (S1110) in the same manner as in the present embodiment (FIG. 11).

After step S1110, the client apparatus 10 executes estimation request (S8111).

Specifically, when the user inputs user identification information into the field object F1110 and operates the operation object B1110, the processor 12 transmits estimation request data to the server 30.

The estimation request data includes, for example, the following information:
User identification information entered in the field object F1110; and
Core temperature information obtained in step S8110.

After step S1111, the server 30 executes calculating DPG parameters (S8130).

Specifically, the processor 32 refers to a core temperature log database (FIG. 5) associated with the user identification information included in the estimation request data to specify the core temperature history for a specified period (for example, 24 hours prior to the date and time of execution of step S1310).

The processor 32 refers to the organism log database (FIG. 9) associated with the user identification information included in the estimation request data to specify the skin temperature history for the specified period (that is, the same period as the core temperature log information).

The processor 32 calculates at least one of the following values as the DPG based on the specified core temperature history and skin temperature history:
Difference between the core temperature and the skin temperature included in the same time window (for example, the time difference between the time stamp information in the core temperature log database (FIG. 5) and the time stamp information in the organism log database (FIG. 9) is within a predetermined time); and
Value obtained by applying a predetermined filter (for example, a smoothing filter configured to reduce noise (for example, a Savitzky-Golay filter)) to the difference between the core temperature and the skin temperature included in the same time window.

In this way, the DPG parameters for each time window are obtained.

As a result, the DPG parameter history (that is, information in which the DPG parameters for each time window are arranged in chronological order) is obtained.

After step S8130, the server 30 executes estimating skin condition (S8131).

Specifically, the memory 31 stores the skin condition model.

The skin condition model describes the correlation between the DPG parameter history and the skin condition.

The skin condition model includes at least one of the current skin condition model and the future skin condition model.

The current skin condition model describes the correlation between the DPG parameter history and the current skin condition.

The future skin condition model describes the correlation between the DPG parameter history and the future skin condition.

The processor 32 inputs the DPG parameters into the current skin condition model to output the current skin condition corresponding to the DPG parameter history. The processor 32 inputs the specified DPG parameters into the future skin condition model to output the future skin condition corresponding to the DPG parameters.

After step S8131, the server 30 executes generating advice (S1131) in the same manner as in the present embodiment (FIG. 11).

After step S8131, the server 30 executes estimating inner body rhythm (S8132).

The inner body rhythm means changes that occur rhythmically in a cycle on the human time axis (for example, 24 hours) when viewed from the perspective of a human being.

In a first example of step S8132, the memory 31 stores a inner body rhythm estimation model.

The inner body rhythm estimation model describes the correlation between the core temperature history and the inner body rhythm.

The processor 32 inputs the core temperature history specified in step S8130 into the inner body rhythm estimation model to output a inner body rhythm corresponding to the core temperature history.

In the second example of step S8132, the memory 31 stores a inner body rhythm estimation model.

The inner body rhythm estimation model describes the correlation between movements during sleep and the inner body rhythm.

The processor 32 acquires sleep movement information on the user's movements during sleep from a device equipped with an acceleration sensor (for example, a wearable device, a smartphone, a pillow, a mattress, or a bed), or an image sensor.

The processor 32 inputs the sleep movement information into the inner body rhythm estimation model to estimate the inner body rhythm according to the movements during sleep.

After step S8131, the server 30 executes estimating organism clock (S8133).

Specifically, the memory 31 stores an organism clock estimation model.

The organism clock estimation model describes the correlation between core temperature history and the organism clock.

The processor 32 inputs the core temperature history specified in step S8130 into the organism clock estimation model to output information (hereinafter referred to as “organism clock information”) about the organism clock corresponding to the core temperature history.

The processor 32 stores the organism clock information in the memory 31 in association with a combination of the user identification information and the execution date and time of step S8133.

After step S8133, the server 30 executes generating DPG advice (S8134).

Specifically, the memory 31 stores a DPG advice model.

The DPG advice model describes the correlation between the DPG parameter history and the DPG advice.

The DPG advice is advice for improving the rhythm of changes in inner body rhythm (for example, increasing the frequency of changes in DPG parameters (that is, increases and decreases in DPG parameters)).

The inner body rhythm means the rhythmic nature of organisms (including organisms other than humans).

There are more than 300 types of the inner body rhythm.

The DPG advice may include, for example, at least one of the following:
Advice on exercise content;
Advice on bathing (for example, at least one of the following: recommended bath time, recommended bath temperature, recommended bath additives (for example, an active ingredient contained in the bath additives and that effectively acts on the core temperature (for example, ginger extract)), and bathing method (for example, a method of using the recommended bath additives));
Advice on sleep-in time;
Advice on wake-up time;
Beauty treatments (for example, facial massage (such as massage of facial muscles) or the like), or treatments using beauty products or beauty devices (for instance, methods of using warming agents); and
Advice on relaxation (for example, using heat pads, steam heaters to warm the face or body, or advice on how to use medicines that have a heating effect).

Processor 32 inputs the DPG parameters obtained in step S8130 into a DPG advice model to output information (hereinafter referred to as “DPG advice information”) about DPG advice.

After step S8134, the server 30 executes updating database (S8135).

Specifically, the processor 32 adds a new record to the core temperature log database (FIG. 5) associated with the user identification information included in the estimation request data.

The following information is stored in each field of the new record:
“time stamp” field: Information about the execution date and time of step S8110; and
“core temperature” field: the core temperature information included in the estimation request data.

After step S8135, the server 30 executes estimation response (S8136).

Specifically, the processor 32 transmits the estimation response data to the client apparatus 10.

The estimation response data includes, for example, the following information:
Core temperature information obtained in step S8130;
Current skin condition information obtained in step S1130;
Future skin condition information obtained in step S1130;
DPG parameter history obtained in step S8130;
Advice information obtained in step S1131;
Estimation result of the inner body rhythm obtained in step S8132;
Organism clock information obtained in step S8133; and
DPG advice information obtained in step S8134.

After step S8136, the client apparatus 10 executes displaying estimation result (S8112).

Specifically, the processor 12 displays screen P8110 (FIG. 21) on the display.

Screen P8110 includes display objects A1111 and A8110a to A8110c, and an image object IMG8110.

The display object A 1111 is the same as that in FIG. 12.

The display object A 8110a is an object that displays the core temperature information obtained in step S8130 (that is, the current core temperature information (at the time of executing the display of the estimation result (S1112))).

The display object A 8110b is an object that displays the image object IMG indicating the current time and the DPG parameter history.

The image object IMG8110 has a circular shape (for example, similar to an analog clock).

The image object IMG8110 has the following areas:
Inner annular area IMG8110a; and
Outer annular area IMG8110b.

The inner annular area IMG8110a is the area that forms the inside of the annular shape.

Similar to an analog clock, numbers indicating the time (for example, 0 to 23), a current time line L8110c, and an internal body time line L8110d are displayed in the inner annular area IMG8110a.

The current time line L8110c indicates the current time (the time when the display of the estimation result (S1112) is executed).

The internal body time line L8110d indicates the time of the organism clock information.

The outer-annular area IMG8110b is the region that forms the outside of the annular shape.

In the outer circular area IMG8110b, a line (hereinafter referred to as the “DPG parameter line”) L8110a indicating the DPG parameter history and a line (hereinafter referred to as the “ inner body rhythm line”) L8110b indicating the inner body rhythm are displayed.

The DPG parameter line L8110a is plotted at a position according to the value of the DPG parameter for each time shown in the outer ring area IMG8110b.

The farther the plot position of the DPG parameter line L8110a is from the center of the inner annular area IMG8110a, the higher the DPG parameter (that is, the greater the difference between the core temperature and the skin temperature).

The inner body rhythm line L8110b represents the life rhythm for each time shown in the outer annular area IMG8110b.

The inner body rhythm line L8110b is plotted at a position according to the value of the inner body rhythm level (for example, the sleep rhythm stored in the action log database (FIG. 10)).

The farther the plot position of the inner body rhythm line L8110b is from the center of the inner annular area IMG8110a, the better the inner body rhythm (for example, the higher the sleep level (that is, the deeper the sleep state)).

The display object A8110c is an object that displays DPG advice information. For example, the DPG advice information is information on the ideal time to take a bath.

### (6-7-3) Summary of Modification 7

According to the seventh modification, the skin condition may be estimated based on the DPG parameters.

This allows for more parameters to be referenced than in the case that DPG parameters are not used, so the accuracy of skin condition estimation can be further improved and advice more suitable for improving skin condition can be presented.

According to the seventh modification, the DPG parameter history may be displayed in the form of a circle.

This allows the rhythm of the DPG parameters to be clearly communicated to the user.

In the seventh modification, the inner body rhythm may be obtained from a wearable device worn by the user.

In this case, estimating inner body rhythm (S8132) can be omitted.

In the seventh modification, the organism clock estimation model may describe the correlation between the organism clock and at least one of the following instead of the core temperature history:
Rhythmicity of gene expression;
Sleep-wake cycle that can be specified through electroencephalogram measurements; and
Urinary steroid hormones that can be specified through blood tests.

### (6-8) Eighth Modification

The eighth modification will now be described.

The eighth modification is an example in which at least one of the current skin condition and the future skin condition, and the inner body rhythm are estimated based on the core temperature history.

### (6-8-1) Overview of Eighth Modification

The overview of the eighth modification will now be described.

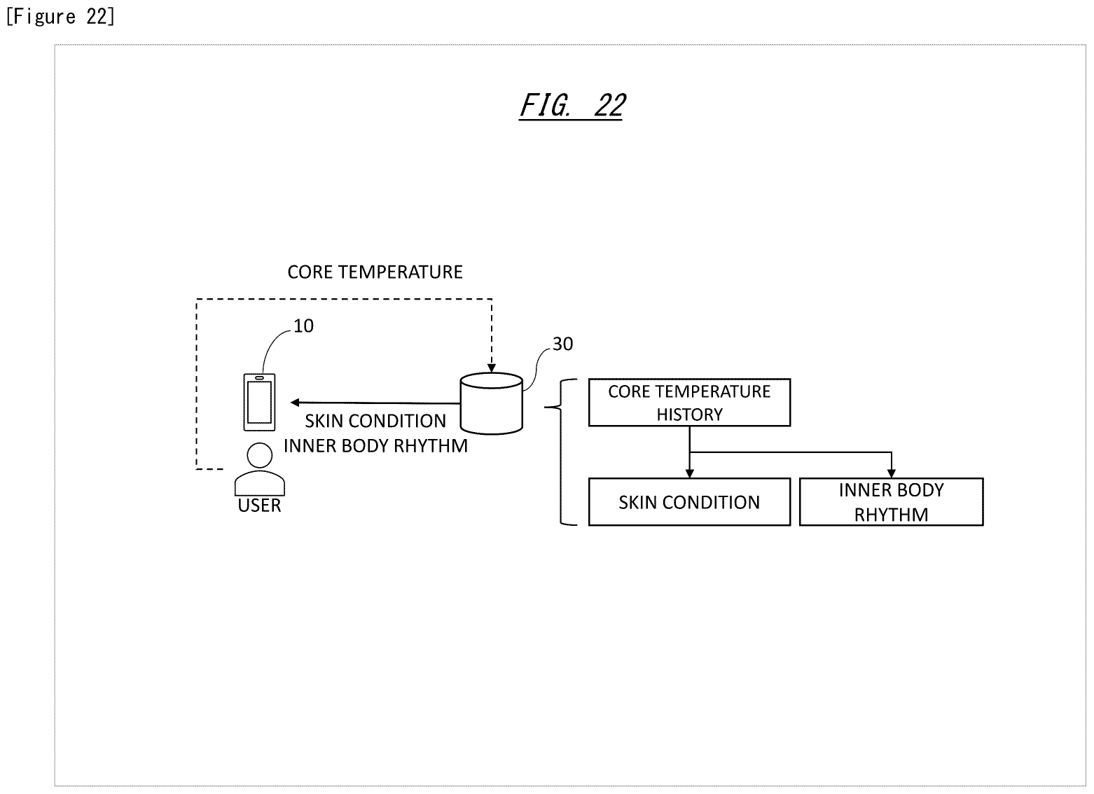

FIG. 22 is a diagram illustrating an overview of the eighth modification.

As shown in FIG. 22, the server 30 stores the user's core temperature history.

The server 30 estimates the user's skin condition and the inner body rhythm based on the core temperature history.

The server 30 presents the estimation results (that is, the estimation results of the user's skin condition and the estimation results of the inner body rhythm) to the user via the client apparatus 10.

### (6-8-2) Information Processing of Eighth Modification

The information processing of the eighth modification will be described.

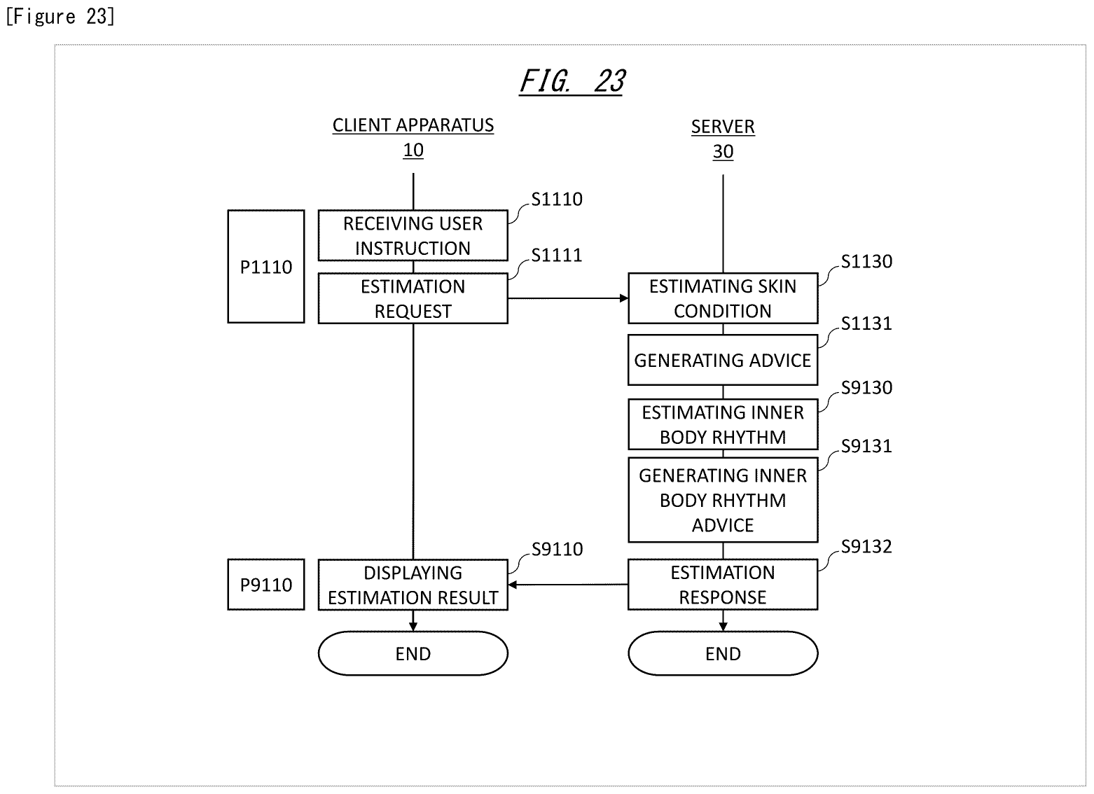

FIG. 23 is a sequence diagram of information processing of the eighth modification.

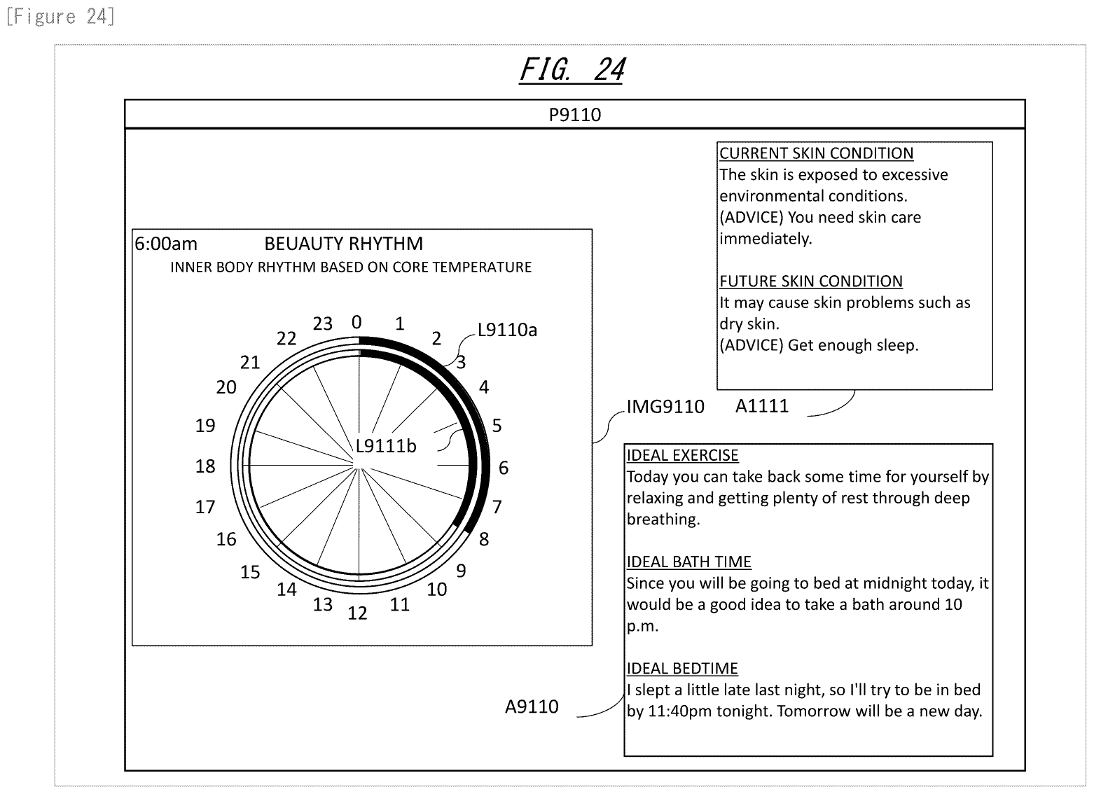

FIG. 24 is a diagram showing an example of a screen displayed in the information processing of FIG. 23.

As shown in FIG. 24, the client apparatus 10 executes the steps from receiving user instruction (S1110) to estimation request (S1111) in the same manner as in the present embodiment (FIG. 11).

After step S1111, the server 30 executes the steps from estimating skin condition (S1130) to generating advice (S1131) in the same manner as in the present embodiment (FIG. 11).

After step S1131, the server 30 executes estimating inner body rhythm (S9130). Specifically, the memory 31 stores an inner body rhythm estimation model.

The inner body rhythm estimation model describes the correlation between the core temperature history and the inner body rhythm.

The inner body rhythm includes, for example, at least one of the following:
Circadian rhythm;
Circaseptan rhythm (that is, weekly rhythm);
Circalunar rhythm;
Circannual rhythm;
Sleep rhythm;
Body temperature rhythm;
Psychogenic stress rhythm;
Heatstroke risk rhythm (for example, the time-series changes in the level of heatstroke danger);
Depression rhythm (for example, changes in depression level over time);
Menstrual rhythm; and
Seasonal rhythm.

The inner body rhythm estimation model includes a real rhythm estimation model and an ideal rhythm estimation model.

The real rhythm estimation model describes the correlation between the core temperature history and real inner body rhythm.

The ideal rhythm estimation model describes the correlation between at least one of the user's place of residence and the action history, and the ideal inner body rhythm.

The processor 32 refers to the core temperature log database (FIG. 5) associated with the user identification information included in the estimation request data to specify the core temperature log information for a specified period (for example, one month prior to the date and time of execution of step S1130).

The processor 32 inputs the specified core temperature log information into the real rhythm model to output information (hereinafter referred to as “real inner body rhythm information”) on the real inner body rhythm corresponding to the core temperature log information.

The real inner body rhythm information is at least one of the following:
Information on the real inner body rhythm for one day (for example, 24 hours prior to the execution date and time of step S1130, or 24 hours prior to the execution date and time of step S1130); and
Information on the average of the real inner body rhythm for n days (n is an integer equal to or greater than 2) (for example, n days prior to the execution date and time of step S1130, or n days prior to the day before the execution date and time of step S1130).

The processor 32 refers to the user database (FIG. 4) associated with the user identification information included in the estimation request data to specify the user's address information.

The processor 32 refers to the action log database (FIG. 10) associated with the user identification information included in the estimation request data to specify the action log information for a predetermined period (for example, one month prior to the execution date and time of step S1130).

The processor 32 inputs at least one of the specified address information and the specified action log information into the ideal rhythm model to output information (hereinafter referred to as “ideal rhythm information”) on the ideal inner body rhythm corresponding to the address information and at least one of the action log information.

The ideal body rhythm is one that has a positive effect on the skin condition.

The temperature where the user spends their time affects the DPG parameters through changes in peripheral skin temperature due to vascular heat release.

For example, if the user's address information indicates a high temperature area, the ideal inner body rhythm will have more fluctuations in the inner body rhythm during cooler hours and less fluctuations in the inner body rhythm during hot hours.

For example, if the user's address information indicates a low temperature area, the ideal inner body rhythm will have less fluctuation in the inner body rhythm during cooler hours and more fluctuation in the inner body rhythm during hot hours.

For example, if the user's action log information indicates that the user is active in the morning, the ideal organism logical rhythm would be one in which the organism logical rhythm fluctuates more in the morning and less in the evening.

For example, if the user's action log information indicates that the user is active at night, the ideal inner body rhythm would be one in which the inner body rhythm fluctuates more at night and less in the morning.

After step S9130, the server 30 executes generating inner body rhythm advice (S9131).

Specifically, the memory 31 stores an inner body rhythm advice model.

The inner body rhythm advice model describes the correlation between the inner body rhythm and the inner body rhythm advice.

The inner body rhythm advice is advice for improving the inner body rhythm so as to create a positive circulation or have a positive effect on at least one of the physical condition (for example, at least one of the skin condition and the inner body condition) and the mental condition.

The inner body rhythm advice includes, for example, at least one of the following:
Advice on exercise;
Advice on bath time;
Advice on sleep time (for example, at least one of wake-up time and sleep-in time);
Advice on rest times;
Advice on beauty action (for example, types of cosmetics, care products, or beauty devices (hereinafter referred to as “beauty products”), how to use beauty products, recommended times for beauty action, and advice on beauty methods (for example, massage methods);
Advice on resting or napping; and
Advice on beauty supplements (for example, those that contain ingredients that have a sweat-inducing or heat-absorbing effect).

The processor 32 inputs the inner body rhythm information obtained in step S9130 into the inner body rhythm advice model to output information (hereinafter referred to as “inner body rhythm advice information”) on the inner body rhythm advice corresponding to the inner body rhythm information.

After step S9131, the server 30 executes estimation response (S9132).

Specifically, the processor 32 transmits the estimation response data to the client apparatus 10.

The estimation response data includes, for example, the following information:
Current skin condition information obtained in step S1130;
Future skin condition information obtained in step S1130;
Advice information obtained in step S1131;
Real inner body rhythm information obtained in step S9130;
Ideal inner body rhythm information obtained in step S9130; and
Inner body rhythm advice information obtained in step S9131.

After step S9132, the client apparatus 10 executes displaying the estimation result (S9110).

Specifically, the processor 12 displays screen P9110 (FIG. 24) on the display.

Screen P9110 includes display objects A1111 and A9110, and an image object IMG9110.

The display object A 1111 is the same as that in FIG. 12.

A display object A 9110 is an object that displays the inner body rhythm advice information.

For example, the inner body rhythm advice information includes information on:
Ideal exercise;
Ideal input time; and
Ideal sleep-in time.

The image object IMG9110 has a circular shape (for example, similar to an analog clock).

The image object IMG9110 displays numbers indicating the time (for example, 0 to 23), an ideal line L9110a, and a real line L9110b, similar to an analog clock.

The ideal line L9110a indicates ideal intracellular rhythm information.

The ideal inner body rhythm information is, for example, an ideal sleep rhythm (for example, sleep time and wakefulness time).

FIG. 24 shows an example in which the ideal sleep time is from midnight to 8:00, and the ideal wake-up time is from 8:00 onwards.

The real line L9110b indicates the real inner body rhythm information.

The real inner body rhythm information is, for example, the real sleep rhythm (as one example, the sleep time and the wakefulness time).

For example, FIG. 24 shows that the actual sleep time is from midnight to 8:00, and the actual wakeful time is from 8:00 onwards.

That is, FIG. 24 shows that the ideal inner body rhythm and the actual inner body rhythm match.

### (6-8-3) Summary of Eighth Modification

According to the eighth modification, the inner body rhythm may be estimated based on the core temperature history.

This makes it possible to further improve the accuracy of estimating the inner body rhythm that affects the skin condition, and also makes it possible to present advice that is more suitable for improving the skin condition.

### (6-9) Ninth Modification

The ninth modification will now be described.

The ninth modification is an example in which, in addition to the core temperature history, time-varying information of the user is presented to the user.

### (6-9-1) Overview of Ninth Modification

The overview of the ninth modification will now be described.

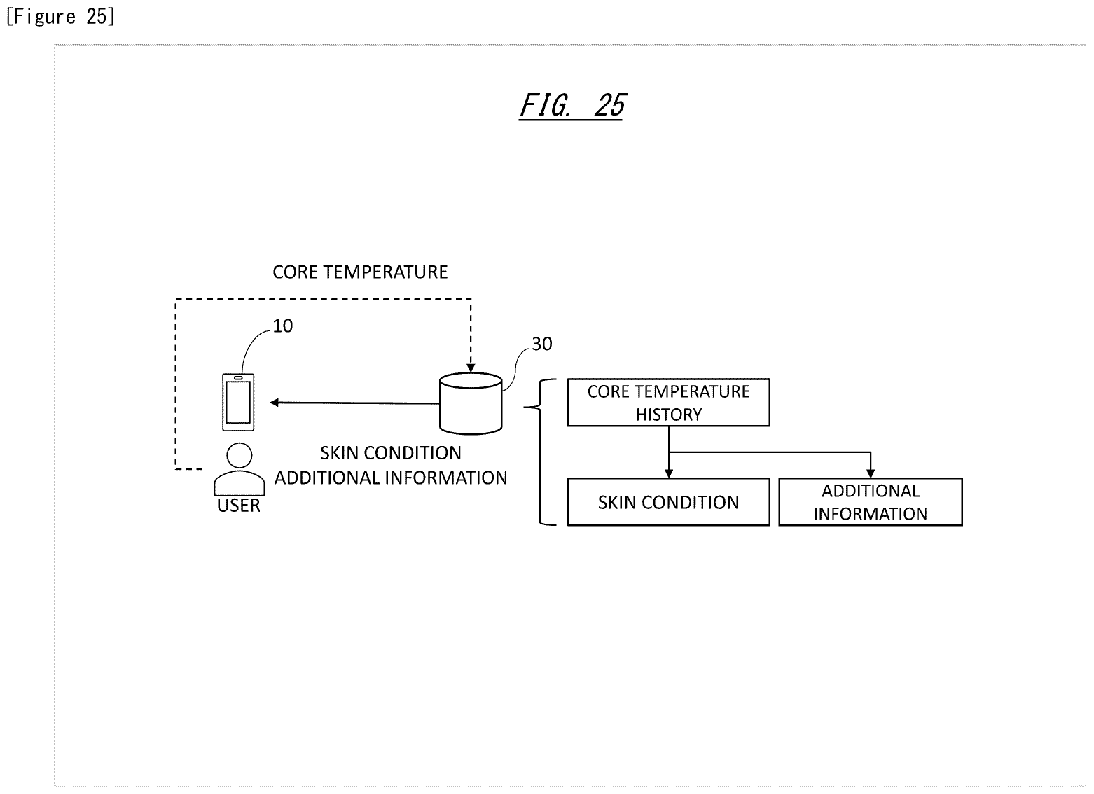

FIG. 25 is a diagram illustrating an overview of the ninth modification.

As shown in FIG. 25, the server 30 stores the user's core temperature history. The server 30 estimates the user's skin condition based on the core temperature history.

The server 30 generates the time displacement information by a method to be described later.

The server 30 presents the estimation result (that is, the estimation result of the user's skin condition) and time displacement information to the user via the client apparatus 10.

The time displacement information includes, for example, at least one of the following:
Skin level information on the user's skin condition level history;
Location information on the user's location history over time;
Environmental information on the user's environment over time (that is, the environment in which the user spent time);
Menstrual cycle information on the user's menstrual cycle history;
Skin age information on the user's skin age history; and
Organism log information of the user.

### (6-9-2) Information Processing of Ninth Modification

The information processing of the ninth modification will be described.

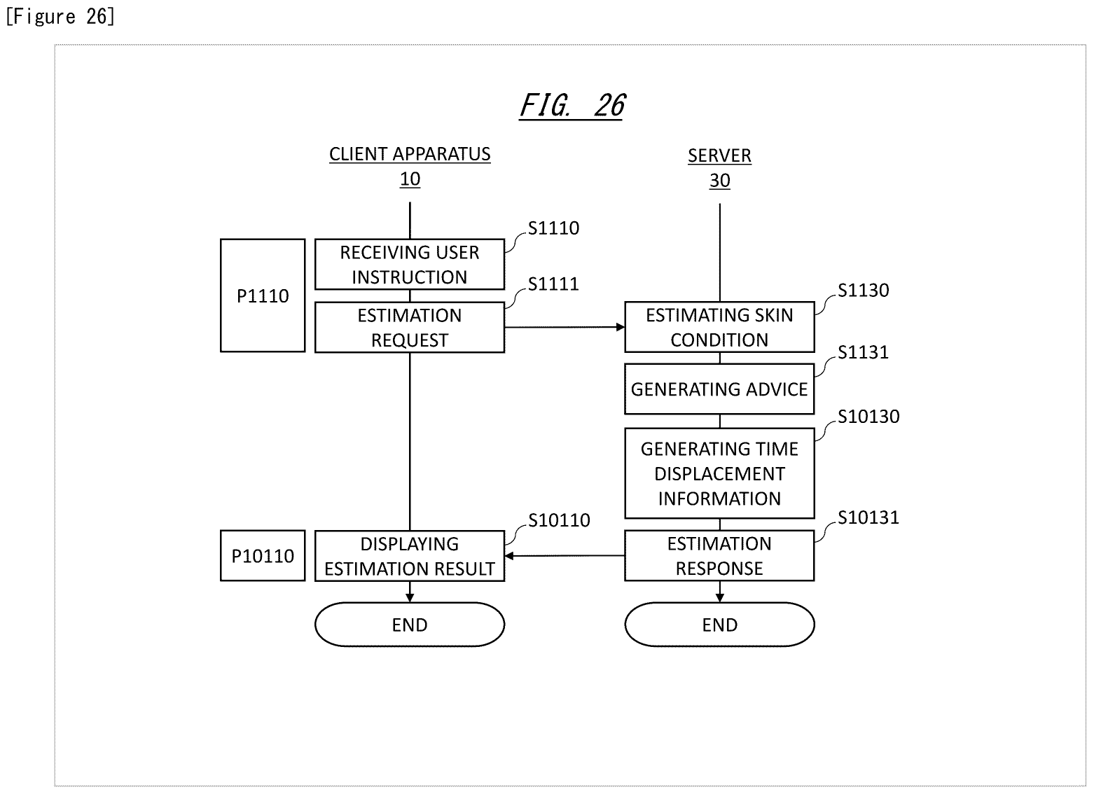

FIG. 26 is a sequence diagram of information processing of the ninth modification.

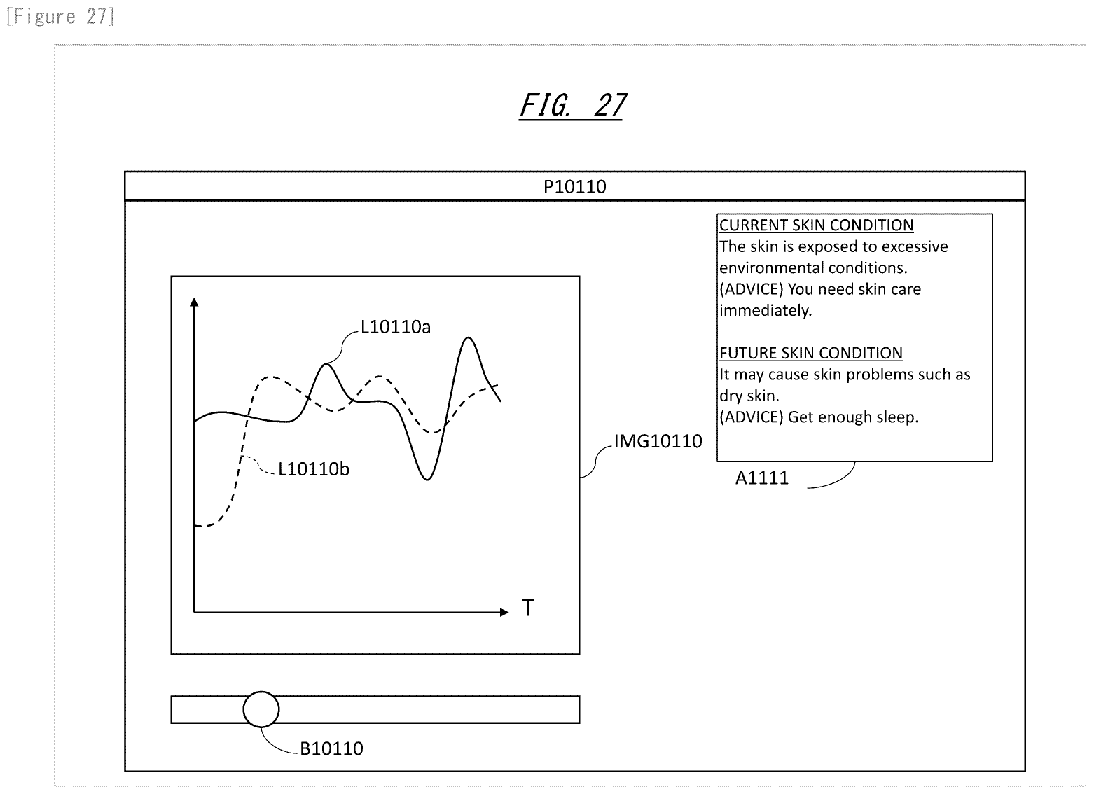

FIG. 27 is a view showing an example of a screen displayed in the information processing of FIG. 26.

As shown in FIG. 26, the client apparatus 10 executes the steps from receiving user instruction (S1110) to estimation request (S1111) in the same manner as in the present embodiment (FIG. 11).

After step S1111, the server 30 executes the steps from estimating skin condition (S1130) to generating advice (S1131) in the same manner as in the present embodiment (FIG. 11).

After step S1131, the server 30 executes generating time displacement information (S10130).

In the first example of step S10130, the server 30 generates skin level information as time displacement information.

Specifically, the skin level information includes at least the following information on the skin condition level history:
Physical condition (for example, skin viscoelasticity, stratum corneum moisture content, stratum corneum barrier function, antioxidant function, sebum amount, blood flow rate, stratum corneum condition, skin color, skin flexibility, glycation level, blood, urine, and sebum RNA); and
Qualitative condition (for example, skin age, skin moisture, skin sagging, skin condition, makeup application, and susceptibility to worsening of skin disorders (for example, acne or rough skin)).

The memory 31 stores the skin condition level determination model.

The skin condition level determination model describes the correlation between the current skin condition and the skin condition level.

The processor 32 inputs the current skin condition obtained in step S1130 into the skin condition level determination model to output skin level information corresponding to the current skin condition.

The processor 32 stores the skin level information in the memory 31 as time displacement information in association with a combination of the user identification information and information on the date and time of execution of step S10130.

In the second example of step S10130, the server 30 generates the position information as the time displacement information.

Specifically, the processor 32 acquires hourly location information from the device carried by the user, and stores the location information in the memory 31 in association with a combination of user identification information and information on the date and time of execution of step S10130.

The device may, for example, include at least one of the following:
Smartphone equipped with GPS (Global Positioning System); and
Wearable device equipped with GPS (for example, a ring-shaped device).

In the third example of step S10130, the server 30 generates environmental information as time displacement information.

Specifically, the processor 32 acquires time-based location information from the device carried by the user, and stores the location information in the memory 31 as time displacement information in association with a combination of user identification information and information on the date and time of execution of step S10130.

The processor 32 acquires environmental information corresponding to the location information from an external server (for example, a server that provides environmental information for each combination of time and location), and stores the environmental information in the memory 31 as time displacement information in association with a combination of user identification information and information on the date and time of execution of step S10130.

The environmental information includes, for example, information on at least one of the following:
Weather;
Temperature;
Humidity;
Ultraviolet exposure; and
Amount of light exposed to the user (hereinafter referred to as “illumination amount”).

In the fourth example of step S10130, the server 30 generates menstrual cycle information as time displacement information.

Specifically, the memory 31 stores a menstrual cycle determination model.

The menstrual cycle determination model describes the correlation between the core temperature history and the menstrual cycle.

The processor 32 refers to the core temperature log database (FIG. 5) associated with the user identification information included in the estimation request data to specify core temperature log information for a specified period (for example, one month prior to the date and time of execution of step S1130).

The processor 32 inputs the specified core temperature log information into a menstrual cycle determination model to output the menstrual cycle corresponding to the core temperature log information.

The processor 32 stores the menstrual cycle in the memory 31 as time displacement information in association with a combination of user identification information and information on the date and time of execution of step S10130.

In the fifth example of step S10130, the server 30 generates skin age information corresponding to the core temperature history as time displacement information.

Specifically, the memory 31 stores a skin age determination model.

The skin age determination model describes the correlation between the core temperature history and skin age.

The processor 32 refers to the core temperature log database (FIG. 5) associated with the user identification information included in the estimation request data to specify the core temperature log information for a specified period (for example, one month prior to the date and time of execution of step S1130).

The processor 32 inputs the specified core temperature log information into the skin age determination model to output a skin age corresponding to the core temperature log information.

The processor 32 stores the skin age in the memory 31 as time displacement information in association with a combination of user identification information and information on the date and time of execution of step S10130.

In the sixth example of step S10130, the server 30 generates organism log information as time displacement information.

Specifically, the processor 32 refers to an organism log database (FIG. 9) associated with the user identification information included in the estimation request data to specify organism log information for a specified period (for example, one month prior to the execution date and time of step S1130).

After step S10130, the server 30 executes estimation response (S10131).

Specifically, the processor 32 transmits the estimation response data to the client apparatus 10.

The estimation response data includes, for example, the following information:
Current skin condition information obtained in step S1130;
Future skin condition information obtained in step S1130;
Advice information obtained in step S1131; and
Time displacement information obtained in step S10130.

After step S10131, the client apparatus 10 executes estimation result (S10110).

Specifically, the processor 12 displays screen P10110 (FIG. 27) on the display.

Screen P10110 includes a display object A1111, an operation object B10110, and an image object IMG10110.

The display object A 111 is the same as that in FIG. 12.

The operation object B10110 is an object (for example, a slider object) that accepts a user instruction for changing the time scale of the image object IMG10110.

Image object IMG10110 is a line graph.

The horizontal axis of the line graph represents time T.

The vertical axis of the line graph represents the core temperature and the time displacement information.

Image object IMG10110 includes the core temperature log line L10110a and the time displacement line L10110b.

The core temperature log line L10110a shows the core temperature history corresponding to the time scale on the horizontal axis.

The time displacement line L10110b indicates the history of time displacement information corresponding to the time scale on the horizontal axis.

When the user operates the operation object B10110, the processor 12 selects the scale of the horizontal axis of the line graph to match the time scale corresponding to the position of the slider, and displays the core temperature log line L10110a and the time displacement line L10110b corresponding to the changed scale.

Time scale options include:
Seconds;
Minutes;
Hour;
Day;
Month; and
Year.

The time displacement information when the time scale is a first time scale (for example, seconds, minutes or hours) is preferably:
Skin level information;
Location information; and
Environmental information.

The time displacement information when the time scale is a second time scale greater than the first time scale (for example, days, months or years) is preferably menstrual cycle.

When the time scale is the largest time scale (for example, year) among the second time scales, the time displacement information is preferably skin age.

### (6-9-3) Summary of Ninth Modification

According to the ninth modification, time displacement information may be presented in addition to the core temperature history.

This allows the user to recognize factors that affect the skin condition (the core temperature history and time-varying information).

### (7) Other Modifications

Other modifications will now be described.

The memory 11 may be connected to the client apparatus 10 via a network NW. The memory 31 may be connected to the server 30 via a network NW.

Each step of the above information processing can be executed by either the client apparatus 10 or the server 30.

For example, if the client apparatus 10 is capable of executing all the steps of the above-mentioned information processing, the client apparatus 10 functions as an information processing apparatus that operates standalone without transmitting requests to the server 30.

In the present embodiment, an example has been shown in which the trigger for the information processing in FIG. 8 is the user's access to a predetermined website using the client apparatus 10, but the present embodiment is not limited to this.

This embodiment is also applicable to an example in which the display of the estimation result (S1112) is executed without a user's instruction.

For example, the client apparatus 10 acquires core temperature information from a wearable sensor and transmits it to the server 30.

The server 30 uses the core temperature information transmitted from the client apparatus 10 to execute the estimation of the organism logical rhythm (S1130) to the estimation response (S1132).

The client apparatus 10 uses the estimation response data transmitted from the server 30 to display the estimation result (S1112).

According to this example, the estimated skin condition is presented to the user in response to acquisition of the core temperature information by the wearable device.

This allows the user to obtain an estimation result of the skin condition according to the core temperature without the burden of providing user instructions.

In the present embodiment, an example has been shown in which the skin condition two weeks after the execution of the skin condition estimation (S1131) is estimated as the future skin condition, but the scope of the present embodiment is not limited to this.

This embodiment can also be applied to an example in which the skin condition at a time point arbitrarily designated by the user is estimated as the future skin condition.

In this case, the future skin condition model describes the correlation between the inner body rhythm, a future time point, and the future skin condition.

When the user specifies any future time point, the processor 32 inputs the inner body rhythm corresponding to the core temperature history and the future time point specified by the user into the future skin condition model to output the future skin condition corresponding to the combination of the inner body rhythm and the future time point specified by the user.

Although the present embodiments of the present invention are described in detail above, the scope of the present invention is not limited to the above embodiments. Further, various modifications and changes can be made to the above embodiments without departing from the spirit of the present invention.

In addition, the above embodiments and modifications can be combined.

### REFERENCE SIGNS LIST

1 Information processing system
10 Client apparatus
11 Memory
12 Processor
13 Input and output interface
14 Communication interface
30 Server
31 Memory
32 Processor
33 Input and output interface
34 Communication interface

1. An information processing apparatus comprising:
an acquiring module configured to acquire core temperature log information on a history of a core temperature of a user;
an estimating module configured to estimate skin condition of the user based on the core temperature log information; and
a presenting module configured to present an estimation result of the skin condition to the user.

2. The apparatus of claim 1, wherein the estimating module estimates future skin condition of the user.

3. The apparatus of claim 2, wherein the future skin condition is a tendency of changes in the skin condition that will occur in the future.

4. The apparatus of claim 1, wherein the estimating module estimates current skin condition of the user.

5. The apparatus of claim 1, further comprising a generating module configured to generate advice according to the estimation result of the skin condition, wherein
the presenting module presents the advice.

6. The apparatus of claim 5, wherein the generating module generates advice according to a combination of the estimation result of the skin condition and the user's preference.

7. The apparatus of claim 6, further comprising an estimating module configured to estimate the user's preferences of the action by referring to action log information of the user.

8. The apparatus of claim 7, wherein the estimating module estimates an action that the user is good at by referring to the action log information of the user, and
the generating module generates advice for encouraging the user to perform the action that the user is good at.

9. The apparatus of claim 7, wherein the estimating module estimates an action that the user is not good at by referring to the action log information of the user, and
the generating module generates advice for encouraging an action other than the action that the user is not good at.

10. The apparatus of claim 6, wherein the estimating module estimates the user's preferences by referring to interview information of the user.

11. The apparatus of claim 6, wherein the estimating module estimates the cosmetic preference of the user by referring to skin care log information of the user.

12. The apparatus of claim 6, wherein the estimating module estimates a preference of an action with a significant organism reaction of the user by referring to action log information and organism log information of the user.

13-17. (canceled)

18. The apparatus of claim 1, wherein the estimating module estimates the skin condition based on a history of a DPG (Distal Proximal-temperature Gradient) parameter.

19. The apparatus of claim 18, wherein the presenting module presents the history of the DPG parameter in a circular form.

20. The apparatus of claim 18, further comprising a generating module configured to generate DPG advice for improving the rhythm of change of the DPG parameters based on the history of the DPG parameter.

21. The apparatus of claim 1, further comprising an estimating module configured to estimate a real inner body rhythm based on the history of the core temperature, wherein
the presenting module presents the real inner body rhythm.

22. The apparatus of claim 21, wherein the estimating module estimates an ideal inner body rhythm based on at least one of place of residence and the history of the action of the user.

23. The apparatus of claim 22, wherein the presenting module presents the real inner body rhythm and the ideal inner body rhythm in a circular form.

24. The apparatus of claim 21, further comprising a generating module configured to generate inner body rhythm advice based on the inner body rhythm,
the inner body rhythm advice being advice for improving the inner body rhythm so as to cause a positive circulation or have a positive effect on at least one of physical condition and mental condition.

25. (canceled)

26. An information processing method using a computer processor executing the steps of:
acquiring core temperature log information on a history of core temperature of a user;
estimating skin condition of the user based on the core temperature log information; and
presenting an estimation result of the skin condition to the user.

27. (canceled)
